(19) United States
(12) Patent Application Publication
(10) Pub. No.: US 2021/0214321 A1
(43) Pub. Date: Jul. 15, 2021

(54) NOVEL REACTIVE BENZOTRIAZOLE UV ABSORBER AND USE THEREOF

(71) Applicant: Everlight Chemical Industrial Corporation, Taipei City (TW)

(72) Inventors: Chih-Wei CHEN, Taoyuan City (TW); Ko-Lun CHEN, Taoyuan City (TW); Yao-Hsing HUANG, Taoyuan City (TW)

(21) Appl. No.: 17/105,674

(22) Filed: Nov. 27, 2020

(30) Foreign Application Priority Data
Jan. 13, 2020 (TW) 109101084

(51) Int. Cl. C07D 249/20; C07D 403/14; C07D 403/12; C09D 5/32; C09D 175/04; G02B 1/14; G02B 5/00

(52) U.S. Cl. CPC C07D 249/20; C07D 403/14; C07D 403/12; G02B 5/003; C09D 175/04; G02B 1/14; C09D 5/32

(57) ABSTRACT

A novel reactive benzotriazole UV absorber and a use thereof are disclosed. The benzotriazole UV absorber is represented by the following formula (I):

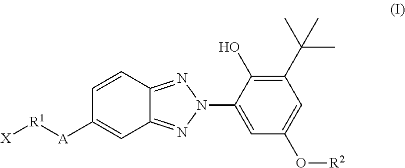

wherein, A, R¹, R² and X are defined in the specification.

### CROSS REFERENCE TO RELATED APPLICATION

This application claims the benefits of the Taiwan Patent Application Serial Number 109101084, filed on Jan. 13, 2020, the subject matter of which is incorporated herein by reference.

### BACKGROUND

### 1. Field

The present disclosure relates to a novel benzotriazole UV absorber with red shifts at the absorption peaks thereof and use thereof. In particular, the present disclosure relates to a novel benzotriazole UV absorber with excellent UV-light absorption property, a composition comprising the same, a glasses lens and a protection film.

### 2. Description of Related Art

An UV absorber is a light stabilizer, which can absorb the UV light in the sun light or fluorescent light. Thus, it is possible to prevent the material contained the UV absorber from being damaged by the UV light. Alternatively, when a coating layer containing the UV absorber is formed on a substrate, it is possible to prevent the substrate from being damaged by the UV light.

Currently, the known UV absorbers mainly can be classified into benzotriazole-based, methyl salicylate-based, benzophenone-based, substituted acrylonitrile-based and triazine-based UV absorbers. Among them, the benzotriazole-based UV absorbers are UV absorbers with high stability. Thus, the material or substrate containing the benzotriazole-based UV absorbers can be prevented from being damaged by the UV light.

However, the absorption ranges of the commercial available benzotriazole-based UV absorbers are still limited to about 400 nm. Thus, it is desirable to provide a novel benzotriazole-based UV absorber, wherein the absorption spectrum thereof shows red shifts at the absorption peaks, and thus the application of the benzotriazole-based UV absorbers can further be extended.

### SUMMARY

An object of the present disclosure is to provide a novel compound, which has excellent UV light absorption property and extraction resistance.

The compound provided by the present disclosure is represented by the following formula (I):

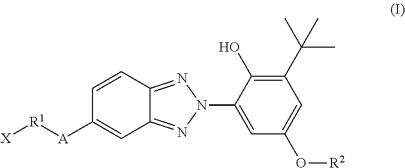

wherein,

A is —S— or —SO₂—;

R¹ is straight or branched C₁₋₁₀ alkylene, straight or branched C₁₋₁₀ alkylene substituted by —OH, or straight or branched C₁₋₁₀ alkylene interrupted by an ester group;

R² is —R³Y, H, straight or branched C₁₋₁₀ alkyl, or C₆₋₁₅ aralkyl;

R³ is straight or branched C₁₋₁₀ alkylene, straight or branched C₁₋₁₀ alkylene substituted by —OH, or straight or branched C₁₋₁₀ alkylene interrupted by an ester group;

each of X and Y respectively is —OH, —OCOR⁴, —NH₂, —NCOR⁴, —NCO,

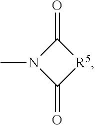

CO₂H, —CO₂R⁴, or

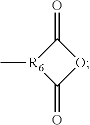

R⁴ is straight or branched C₁₋₁₀ alkyl, or straight or branched C₃₋₁₀ alkenyl;

R⁵ is C₃₋₁₀ cycloalkenylene; and

R⁶ is straight or branched C₁₋₁₀ alkylene, or 1,2-phenylene.

The novel compound provided by the present disclosure is a benzotriazole compound, which can be used as an UV absorber. Herein, a sulfur-containing group is located at the 5-position on the benzo ring of the formula (I), and an oxygen-containing group is located at the 5-position on the phenyl group of the formula (I). By the synergy effect of the sulfur-containing group and the oxygen-containing group, the spectrum of the compound shows a red shift at the absorbance peaks, and thus the compound has an extended absorption range. In particular, the novel compound provided by the present disclosure has an absorption range extended to 450 nm or more, and also has the absorbance peaks at 280 nm to 340 nm which is the conventional absorbance peaks of the UV absorber. Thus, the compound of the present application can be applied to anti-blue light materials for a product such as lens, contacts, etc., and can further be applied to carbon fiber complex materials for aerospace and transportation. In addition, the novel compound of the present disclosure contains a reactive group, and can be polymerized with monomers or oligomers to form polymers with UV absorbers. The UV absorbers in the obtained polymer show excellent extraction resistance.

In the present disclosure, A can be —S— or —SO₂—.

In the present disclosure, R¹ can be straight or branched C₁₋₁₀ alkylene, straight or branched C₁₋₁₀ alkylene substituted by —OH, or straight or branched C₁₋₁₀ alkylene interrupted by an ester group. In one embodiment of the present disclosure, R¹ can be straight or branched C₁₋₆ alkylene, straight or branched C₁₋₃ alkylene substituted by —OH, or straight or branched C₁₋₃ alkylene interrupted by an ester group. In another embodiment of the present disclosure, R¹ can be straight or branched C₁₋₃ alkylene, straight or branched C₁₋₃ alkylene substituted by —OH or straight or branched C₁₋₃ alkylene interrupted by an ester group.

In the present disclosure, R² can be —R³Y, H, straight or branched C₁₋₁₀ alkyl, or C₆₋₁₅ aralkyl. In one embodiment of the present disclosure, R² can be —R³Y, H, straight or branched C₁₋₆ alkyl, or benzyl. In another embodiment of the present disclosure, R² can be —R³Y, H, straight or branched C₁₋₄ alkyl, or benzyl.

In the present disclosure, R³ can be straight or branched C₁₋₁₀ alkylene, straight or branched C₁₋₁₀ alkylene substituted by —OH, or straight or branched C₁₋₁₀ alkylene interrupted by an ester group. In one embodiment of the present disclosure, R³ can be straight or branched C₁₋₆ alkylene, straight or branched C₁₋₆ alkylene substituted by —OH, or straight or branched C₁₋₆ alkylene interrupted by an ester group. In another embodiment of the present disclosure, R³ can be straight or branched C₁₋₃ alkylene, straight or branched C₁₋₃ alkylene substituted by —OH, or straight or branched C₁₋₃ alkylene interrupted by an ester group.

In the present disclosure, each of X and Y respectively can be —OH, —OCOR⁴, —NH₂, —NCOR⁴, —NCO,

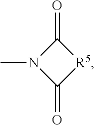

—CO₂H, —CO₂R⁴, or

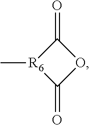

wherein R⁴ can be straight or branched C₁₋₁₀ alkyl, or straight or branched C₃₋₁₀ alkenyl; R⁵ can be C₃₋₁₀ cycloalkenylene; and R⁶ can be straight or branched C₁₋₁₀ alkylene, or 1,2-phenylene. In one embodiment of the present disclosure, each of X and Y respectively can be —OH, —OCOR⁴, —NH₂,

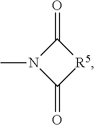

—CO₂R⁴, or

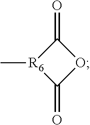

R⁴ can be straight or branched C₁₋₆ alkyl, or straight or branched C₃₋₆alkenyl; R⁵ can be C₃₋₁₀ cycloalkenylene; and R⁶ can be straight or branched C₁₋₆alkylene. In another embodiment of the present disclosure, each of X and Y respectively can be —OH, —OCOR^{4a}, —NH₂,

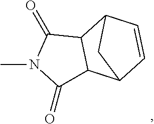

—CO₂R^{4b} or

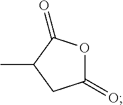

R^{4a} can be straight or branched C₃₋₆ alkenyl; and R^{4b} can be straight or branched C₁₋₆ alkyl.

In one embodiment of the present disclosure, A can be —S—; R¹ can be straight or branched C₁₋₁₀ alkylene, or straight or branched C₁₋₁₀ alkylene substituted by —OH; X can be —OH,

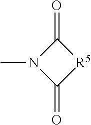

or —CO₂R⁴; R⁴ can be straight or branched C₁₋₁₀ alkyl; and R⁵ can be C₃₋₁₀ cycloalkenylene. In another embodiment of the present disclosure, A can be —S—; R¹ can be straight or branched C₁₋₆ alkylene, or straight or branched C₁₋₆ alkylene substituted by —OH; X can be —OH,

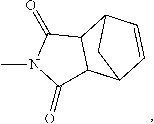

or —CO₂R⁴; R⁴ can be straight or branched C₁₋₆ alkyl. In another embodiment of the present disclosure, A can be —S—; R¹ can be straight or branched C₁₋₄ alkylene, or straight or branched C₁₋₄ alkylene substituted by —OH; X can be —OH.

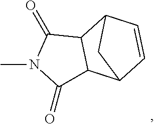

or —CO₂R⁴; R⁴ can be straight or branched C₁₋₄ alkyl.

In one embodiment of the present disclosure, A can be —SO₂—; R¹ can be straight or branched C₁₋₁₀ alkylene, or straight or branched C₁₋₁₀ alkylene interrupted by an ester group; X can be —OH, —OCOR⁴, —NH₂ or

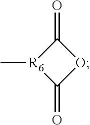

R⁴ can be straight or branched C₃₋₁₀ alkenyl; and R⁶ can be straight or branched C₁₋₁₀ alkylene. In another embodiment of the present disclosure, A can be —SO₂—; R¹ can be straight or branched C₁₋₆ alkylene, or straight or branched C₁₋₆ alkylene interrupted by an ester group; X can be —OH, —OCOR⁴, —NH₂ or

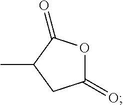

and R⁴ can be straight or branched C₃₋₆ alkenyl. In another embodiment of the present disclosure, A can be —SO₂—; R¹ can be straight or branched C₁₋₄ alkylene, or straight or branched C₁₋₄ alkylene interrupted by an ester group; X can be —OH, —OCOR⁴, —NH₂ or

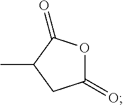

and R⁴ can be straight or branched C₃₋₄ alkenyl.

In one embodiment of the present disclosure, R² can be H, straight or branched C₁₋₁₀ alkyl, or C₆₋₁₅ aralkyl. In another embodiment of the present disclosure, R² can be H, straight or branched C₁₋₆ alkyl, or benzyl. In another embodiment of the present disclosure, R² can be H, straight or branched C₁₋₄ alkyl, or benzyl.

In one embodiment of the present disclosure, R² can be —R³Y; R³ can be straight or branched C₁₋₁₀ alkylene, or straight or branched C₁₋₁₀ alkylene interrupted by an ester group; Y can be —OH, —OCOR⁴, —NH₂,

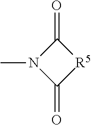

R⁴ can be straight or branched C₃₋₁₀ alkenyl; R can be C₃₋₁₀ cycloalkenyene; and R⁶ can be straight or branched C₁₋₁₀ alkylene. In another embodiment of the present disclosure, R² can be —R³Y; R³ can be straight or branched C₁₋₆ alkylene, or straight or branched C₁₋₆ alkylene interrupted by an ester group; Y can be —OH, —OCOR⁴, —NH₂,

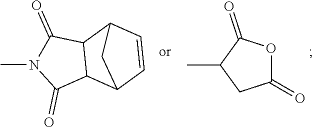

and R⁴ can be straight or branched C₃₋₆ alkenyl. In another embodiment of the present disclosure, R² can be —R³Y; R³ can be straight or branched C₁₋₃ alkylene, or straight or branched C₁₋₃ alkylene interrupted b an ester group; Y can be —OH, —OCOR⁴, —NH₂,

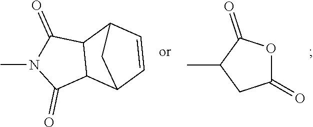

and R⁴ can be straight or branched C₃₋₄ alkenyl.

The compound provided by the present disclosure can be any one of the following formulas (I-1) to (I-13):

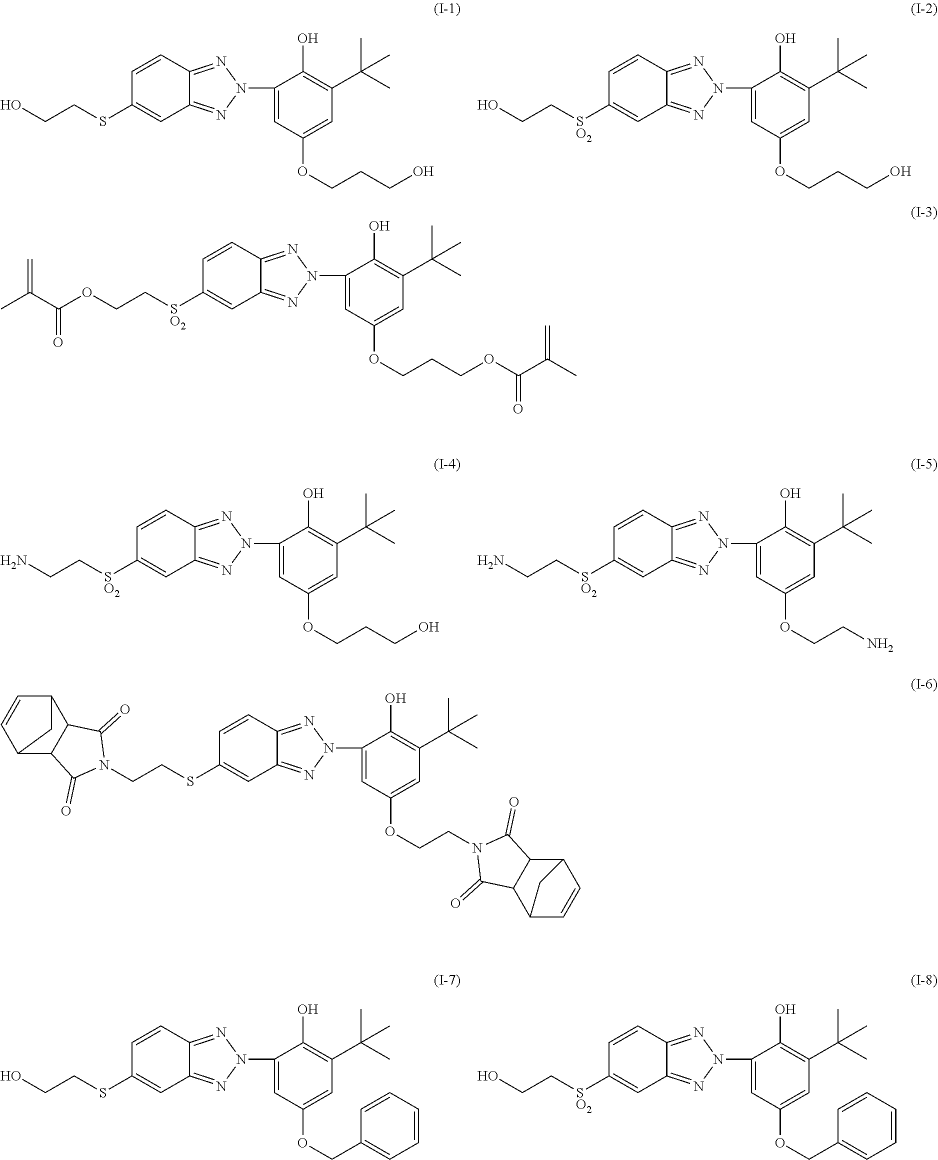

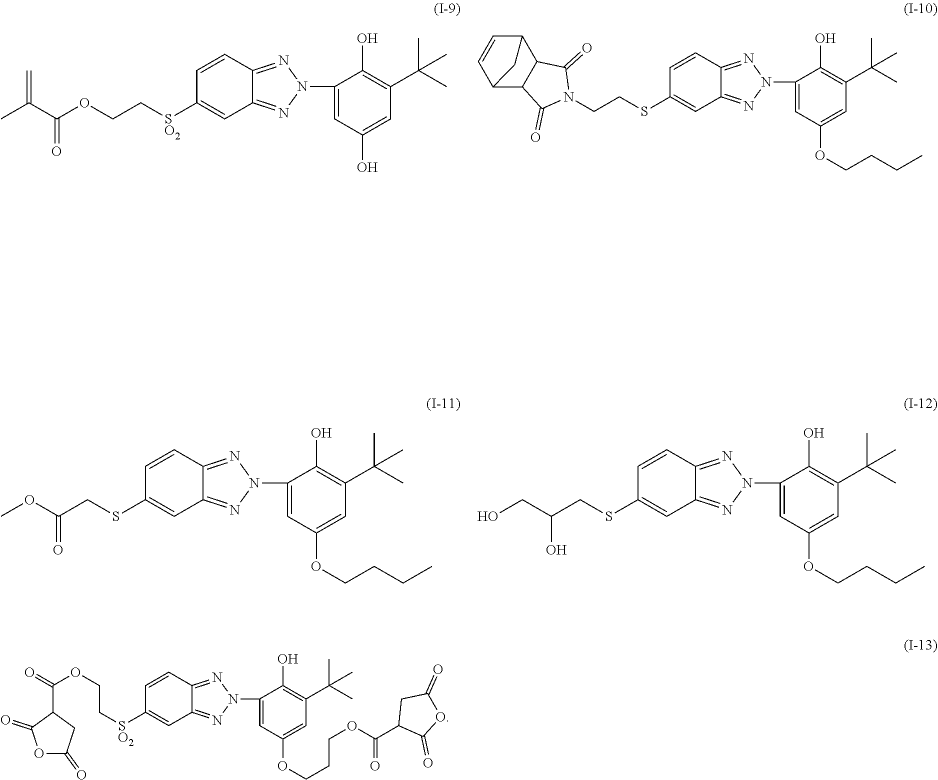

In the present disclosure, the term “alkyl(ene)” refers to straight and branched alkyl(ene), and includes, for example, straight or branched C₁₋₁₀ alkyl(ene), C₁₋₆ alkyl(ene) or C₁₋₄ alkyl(ene). Specific examples of alkyl(ene) include, but are not limited to, methyl(ene), ethyl(ene), n-propyl(ene), iso-propyl(ene), n-butyl(ene), sec-butyl(ene), iso-butyl(ene), tert-butyl(ene), pentyl(ene), neo-pentyl(ene) or hexyl(ene).

In the present disclosure, the term “alkenyl” includes straight or branched hydrocarbon groups with at least one double bond, and includes, for example, straight or branched C₃₋₁₀ hydrocarbon groups with at least one double bond, straight or branched C₃₋₆ hydrocarbon groups with at least one double bond, or straight or branch C₃₋₄ hydrocarbon groups with at least one double bond. Examples of the alkenyl include, but are not limited to propenyl or butenyl.

In the present disclosure, the term “cycloalkenyl(ene)” includes cyclic unsaturated hydrocarbon groups, which includes 3 to 10 carbon atoms (C₃₋₁₀), 5 to 8 carbon atoms (C₅₋₈) or 5 to 7 carbon atoms (C₅₋₇). Examples of the cycloalkenyl(ene) include, but are not limited to cyclopentenyl(ene), cyclohexenyl(ene) or cycloheptenyl(ene).

In the present disclosure, the term “aryl” includes 6-membered single aromatic ring, 10-membered double aromatic ring or 14-membered triple aromatic ring. Examples of the aryl include, but are not limited to phenyl, naphthyl, pyrenyl, anthryl or phenanthryl.

In the present disclosure, the term “aralkyl” refers to a moiety that the alkyl defined in the present disclosure coupled with at least one aryl.

In the present disclosure, the term “alkylene interrupted by an ester group” refers to a moiety that an ester group is introduced between two adjacent carbon atoms of the alkylene defined in the present disclosure, or an ester group is connected to one end of the alkylene defined in the present disclosure. In one embodiment of the present disclosure, “alkylene interrupted by an ester group” can be —C(═O)O-(alkylene).

Furthermore, the present disclosure also provides a composition with stability to photo-induced degradation, which comprises: (A) a photo-induced degradable organic material; and (B) the aforesaid novel compound of the present disclosure. Herein, a content of the novel compound of the present disclosure is 0.1% to 30% based on a weight of the photo-induced degradable organic material.

In one embodiment of the present disclosure, the aforesaid composition is used for forming a coating layer. In particular, the coating layer is formed on a substrate which is sensitive to electromagnetic radiation with a wavelength greater than 380 nm. The material of the substrate is not particularly limited, and can be glass, plastic, polymer, silicone hydrogel, resin, carbon fiber complex material, or a combination thereof.

Furthermore, the present disclosure also provides a glasses lens with anti-blue light effect, which comprises the aforesaid novel compound of the present disclosure. Herein, the novel compound of the present disclosure is applied to a substrate for the glasses lens to form an anti-UV or anti-blue light coating thereon.

In addition, the the present disclosure further provides a protection film with anti-blue light effect, which comprises the aforesaid novel compound of the present disclosure. Herein, the novel compound of the present disclosure is applied to a substrate for the protection film to form an anti-UV or anti-blue light coating thereon. An example of the protection film can be a screen protector, but the present disclosure is not limited thereto.

Other novel features of the disclosure will become more apparent from the following detailed description when taken in conjunction with the accompanying drawings.

### BRIEF DESCRIPTION OF THE DRAWINGS

FIG. 1 is absorption spectra of compounds of Examples 1-2 and Comparative examples 1-2 (Comp exs. 1-2).

FIG. 2 is absorption spectra of compounds of Examples 2, 3 and 8.

FIG. 3 shows transmission results of compounds of Example 2 and Comparative example 1 (Comp ex. 1).

### DETAILED DESCRIPTION OF EMBODIMENT

The following embodiments when read with the accompanying drawings are made to clearly exhibit the above-mentioned and other technical contents, features and/or effects of the present disclosure. Through the exposition by means of the specific embodiments, people would further understand the technical means and effects the present disclosure adopts to achieve the above-indicated objectives. Moreover, as the contents disclosed herein should be readily understood and can be implemented by a person skilled in the art, all equivalent changes or modifications which do not depart from the concept of the present disclosure should be encompassed by the appended claims.

Unless specified otherwise, term “or” used in the present specification and claims includes meaning of “and/or”.

The present disclosure is explained by the following embodiments, which are not used to limit the scope of the present disclosure. Unless specified otherwise, “%” used herein for indicating the amount of the contents or the objects in the following embodiments are weight percentage.

### Preparation Example 1: Synthesis of the Raw Compound 1a

### Preparation of Compound 1a′

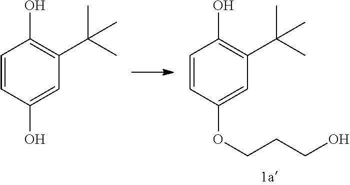 [SMILES: CC(C)(C)c1cc(O)ccc1O ; CC(C)(C)c1cc(OCCCO)ccc1O]

Tertiary butylhydroquinone (856.8 g) was dissolved in methanol (2.5 L), followed by adding NaOH (206.2 g), water (1 L), chloropropanol (482.3 g) and KI (6.3 g). The mixture was heated at reflux temperature under N₂ atmosphere for 48 hr. After the mixture was cooled down to room temperature, water (5.6 L) was added to dilute the mixture, and then the mixture was extracted with dichloromethane (4 L). The combined dichloromethane layer was washed with water, dried with anhydrous sodium sulfate and concentrated. The residue was purified with reduced pressure distillation, and the distillate was recrystallized with toluene for further purification to obtain white solids (396.0 g, m.p. 78.5-80.5° C.).

### Preparation of Compound 1a

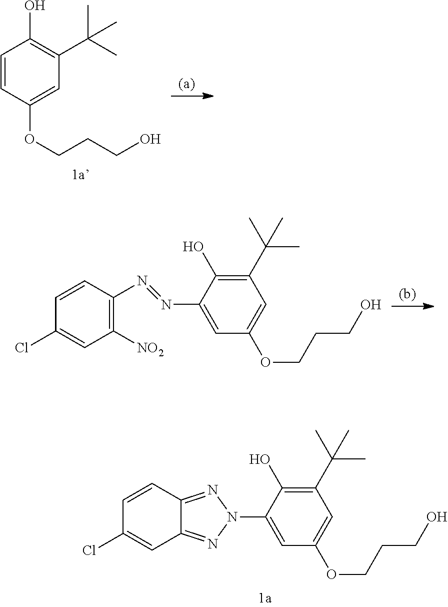 [SMILES: CC(C)(C)c1cc(OCCCO)ccc1O ; CC(C)(C)c1cc(OCCCO)cc(/N=N/c2ccc(Cl)cc2[N+](=O)[O-])c1O ; CC(C)(C)c1cc(OCCCO)cc(-n2nc3ccc(Cl)cc3n2)c1O]

### Step (a)

4-Chloro-2-nitroaniline (90.6 g) and concentrated HCl (180 mL) was mixed and stirred at room temperature for 1 hr, and the mixture was diluted with water (160 mL) and ice (300 g). At −5° C., sodium nitrite (37.7 g) was dissolved in water (140 mL) and then added into the mixture. After adding sodium nitrite, the mixture was stirred at 0° C. for 1 hr, and then sulfamic acid was added therein until the result on the potassium iodide-starch test paper showed negative. Then, the mixture was filtered, the filtrate was added to a mixture containing the compound 1a′ (112.2 g), NaOH (60 g) and water (2 L) at −5° C. to 0° C. under stirring. After adding about ⅓ diazonium solution, 10% NaOH aqueous solution (400 mL) was added together with the diazonium solution into the mixture, and the addition of the diazonium solution and the NaOH aqueous solution was finished at the same time. The addition of the diazonium solution and the NaOH aqueous solution was held at 0° C. or less. After addition, the mixture was stirred for further 2 hr at the same temperature. Then, the mixture was placed to let the temperature of the mixture back to room temperature. The azo dye was separated by acidification with HCl, filtered and washed with water. The obtained azo dye was directly used in the next step without further purification.

### Step (b)

The azo dye obtained in the step (a) was dissolved in ethanol (1.5 L), and a glucose solution (glucose (180 g) dissolved in 2 N NaOH aqueous solution (1.5 L)) was slowly added into the azo dye solution. The temperature was kept at 30° C. or less, and the thin layer chromatography was used to trace the completion of the reaction. Then, fresh activated Zn powders (165 g) were added. The mixture was stirred at room temperature for 3 hr, diluted with water (1 L) and stirred for 15 min, and then left to stand for 1 hr. The precipitate was filtered and separated, followed by washing with water. The filter cake was further extracted with hot ethanol (2 L) until only Zn powders were left in the filter cake. The extract was cooled down to room temperature, and the solid was filtered and separated, followed by washing with cold ethanol. After vacuum drying, yellow solids can be obtained (67.8 g, m.p. 134.7° C.).

¹H NMR (400 MHz, CDCl₃): δ 11.35 (1H), 7.92 (1H), 7.87 (1H), 7.80 (1H), 7.43 (1H), 7.02 (1H), 4.20 (2H), 3.91 (2H), 2.10 (2H), 1.80 (1H), 1.49 (9H) TGA (5% weight loss): 246.2° C.

### Example 1: Synthesis of Compound 2a (i.e. the Compound (I-1) of the Present Disclosure)

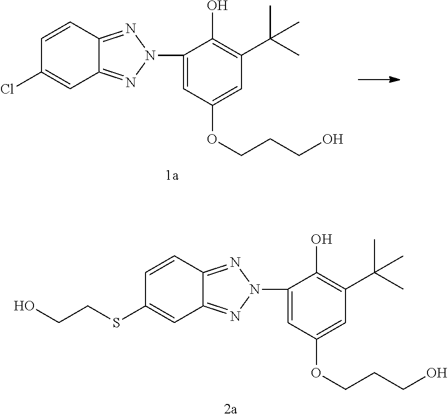 [SMILES: CC(C)(C)c1cc(OCCCO)cc(-n2nc3ccc(Cl)cc3n2)c1O ; CC(C)(C)c1cc(OCCCO)cc(-n2nc3ccc(SCCO)cc3n2)c1O]

To a flask (20 mL), the compound 1a (0.75 g), KOH (0.52 g), KI (0.09 g), 2-mercaptoethanol (0.5 mL), N-methylpyrrolidone (2 mL) were added, and the mixture was heated to 100° C. and stirred for 12 hr. After the mixture was cooled down to room temperature, the mixture was acidified with 1N HCl aqueous solution to pH 5. The mixture was extracted with toluene (100 mL), ethyl acetate (50 mL) and water (100 mL). The water layer was removed, and the organic layer was dried with MgSO₄ to remove water. After reduced pressure concentration and purification with column chromatography, the compound 2a was obtained (0.21 g, 25.2%).

¹H NMR (CDCl₃, 400 MHz): 1.49 (s, 9H), 1.86 (s, 1H), 2.10 (quint, J=5.9 Hz, 2H), 3.25 (t, J=5.8 Hz, 2H), 3.80-3.95 (m, 4H), 4.20 (t, J=6.0 Hz, 2H), 7.06 (d, J=3.0 Hz, 1H), 7.42 (dd, J=1.4, 8.8 Hz, 1H), 7.79 (d, J=3.0 Hz, 1H), 7.82 (d, J=8.8 Hz, 1H), 7.83 (d, J=1.4 Hz, 1H), 11.42 (s, 1H);

¹³C NMR (CDCl₃, 100 MHz): 29.5, 32.2, 35.8, 36.9, 60.5, 60.8, 103.1, 115.8, 117.2, 118.1, 125.3, 129.9, 136.0, 141.1, 141.6, 143.3, 143.6, 151.2; HRMS(ESI⁺, M+H): calc. 418.1801, found 418.1804.

### Example 2: Synthesis of Compound 2b (i.e. the Compound (I-2) of the Present Disclosure)

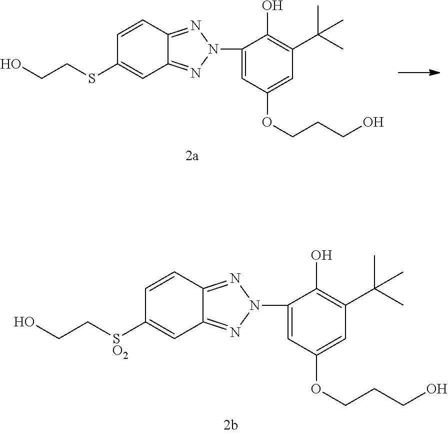 [SMILES: CC(C)(C)c1cc(OCCCO)cc(-n2nc3ccc(SCCO)cc3n2)c1O ; CC(C)(C)c1cc(OCCCO)cc(-n2nc3ccc(S(=O)(=O)CCO)cc3n2)c1O]

To a flask (250 mL), the compound 2a (0.75 g), Na₂WO₄.2H₂O (0.1 g), 90% formic acid aqueous solution (9 mL), toluene (50 mL), 30% H₂O₂ aqueous solution (9 mL) were added, and the mixture was heated to 70° C. and stirred for 6 hr. After the mixture was cooled down to room temperature, the mixture was extracted with toluene (100 mL) and water (10 mL). The water layer was further extracted with toluene (100 mL) and combined with the organic layer. The organic layer was washed with saturated brine (100 mL) and dried with MgSO₄, followed by reduced pressure concentration and purification with chromatography to obtain the compound 2b (0.23 g, 28.3%).

¹H NMR (CDCl₃, 400 MHz): 1.49 (s, 9H), 2.04-2.17 (m, 2H), 2.80 (s, 1H), 3.47 (dt, J=6.0, 2.4 Hz, 2H), 3.92 (t, J=6.0 Hz, 2H), 4.09 (t, J=5.2 Hz, 2H), 4.21 (t, J=6.0 Hz, 2H), 7.07 (d, J=3.2 Hz, 1H), 7.83 (d, J=3.2 Hz, 1H), 7.94 (d, J=9.1 Hz, 1H), 8.12 (dd, J=0.8, 9.1 Hz, 1H), 8.66 (d, J=0.8 Hz, 1H), 11.22 (s, 1H); ¹³C NMR (CDCl₃, 100 MHz): 29.5, 32.2, 35.9, 56.5, 58.6, 60.5, 66.5, 103.2, 118.6, 119.5, 120.8, 125.0, 138.3, 141.5, 141.7, 144.1, 144.4, 151.4; HRMS(ESI⁺, M+H): calc. 450.1699, found 450.1710.

### Example 3: Synthesis of Compound 2c (i.e. the Compound (I-3) of the Present Disclosure)

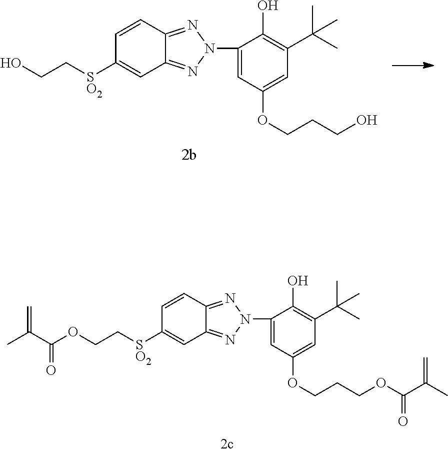 [SMILES: CC(C)(C)c1cc(OCCCO)cc(-n2nc3ccc(S(=O)(=O)CCO)cc3n2)c1O ; C=C(C)C(=O)OCCCOc1cc(-n2nc3ccc(S(=O)(=O)CCOC(=O)C(=C)C)cc3n2)c(O)c(C(C)(C)C)c1]

To a flask (250 mL), the compound 2b (0.85 g), hydroquinone (0.1 g), p-toluenesulfonic acid (0.1 g), methacrylic acid (0.5 mL) and toluene (125 mL) were added, and the mixture was heated and stirred for 16 hr under reflux. After the mixture was cooled down to room temperature, the mixture was extracted with saturated NaHCO₃ aqueous solution (100 mL). The water layer was further extracted with toluene (50 mL) and combined with the organic layer. The organic layer was washed with saturated brine (100 mL) and dried with MgSO₄. After reduced pressure concentration and purification with chromatography, the product was recrystallized with methanol/toluene to obtain the compound 2c (0.34 g, 30.7%).

¹H NMR (CDCl₃, 400 MHz): 1.50 (s, 9H), 1.71 (d, J=1.4 Hz, 3H), 1.97 (d, J=1.4 Hz, 3H), 2.23 (quint, J=6.2 Hz, 2H), 3.63 (t, J=6.0 Hz, 2H), 4.17 (t, J=6.0 Hz, 2H), 4.40 (t, J=6.2 Hz, 2H), 4.56 (t, J=5.8 Hz, 2H), 5.37 (dd, J=0.9, 1.4 Hz, 1H), 5.58 (dd, J=0.9, 1.4 Hz, H), 5.75 (s, 1H), 6.14 (s, 1H), 7.08 (d, J=2.8 Hz, 1H), 7.81 (d, J=2.8 Hz, 1H), 7.93 (dd, J=1.2 Hz, 9.2 Hz, 1H), 8.11 (d, J=9.2 Hz, 1H), 8.66 (d, J=1.2 Hz, 1H), 11.24 (s, 1H); ¹³C NMR (CDCl₃, 100 MHz): 18.1, 18.5, 28.8, 29.5, 35.9, 55.4, 58.0, 61.7, 65.3, 103.0, 119.0, 119.4, 121.0, 124.9, 125.1, 125.7, 126.6, 135.3, 136.5, 138.7, 141.6, 144.1, 144.4, 151.5, 166.6, 167.5; HRMS(ESI⁺, M+H): calc. 586.2223, found 586.2227.

### Example 4: Synthesis of Compound 2d (i.e. the Compound (I-4) of the Present Disclosure)

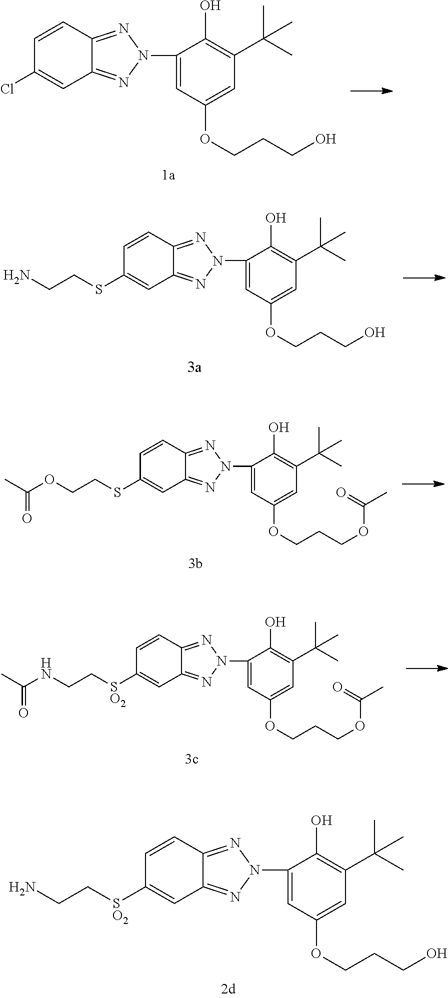

To a flask (25 mL), the compound 1a (0.3 g), K₂CO₃ (2.0 g), 2-aminoethanethiol (1.2 g) and N-methylpyrrolidone (2 mL) were added, the mixture was heated to 100° C. and stirred for 12 hr. After cooling down to room temperature, the mixture was added into water (100 mL), and 1N HCl aqueous solution was added therein until the pH value of the mixture was 5. Isopropanol (25 mL), toluene (75 mL) and NaCl (10 g) was added into the mixture, followed by extraction. The water layer was extracted with isopropanol (25 mL) and toluene (75 mL) and combined with the organic layer. The organic layer was washed with saturated brine (100 mL), followed by reduced pressure concentration and purification with chromatography to obtain an intermediate 3a, which was all used in the sequential reaction.

¹H NMR (Pyridine-d⁵, 400 MHz): 1.59 (s, 9H), 2.29 (quint, J=6.2 Hz, 2H), 3.82 (t, J=7.1 Hz, 2H), 4.05 (t, J=7.1 Hz, 2H), 4.13 (t, J=6.2 Hz, 2H), 4.37 (t, J=6.2 Hz, 2H), 7.24 (d, J=2.6 Hz, 1H), 7.55 (d, J=9.0 Hz, 1H), 7.90 (d, J=9.0 Hz, 1H), 8.00 (d, J=2.6 Hz, 1H), 8.19 (s, 1H); HRMS (ESI⁺, M+H): calc. 417.1960, found 417.1971.

The intermediate 3a was mixed with ethyl acetate (30 mL), tert-butanol (30 mL), acetic anhydride (3 mL) and triethylamine (3 mL), and the mixture was heated to 70° C. and stirred. After the intermediate 3a was dissolved completely, the mixture was cooled down to room temperature. The mixture was extracted with water (100 mL) and ethyl acetate (100 mL), and the water layer was further extracted with ethyl acetate (50 mL) and combined with the organic layer. The organic layer was washed with saturated brine (50 mL), followed by reduced pressure concentration to obtain an intermediate 3b, which was all used in the sequential reaction.

The intermediate 3b was mixed with Na₂WO₄.2H₂O (0.1 g), 30% H₂O₂ aqueous solution (15 mL), water (5 mL) and isopropanol (50 mL), and the mixture was stirred at room temperature for 16 hr. The mixture was extracted with water (100 mL) and ethyl acetate (100 mL), and the water layer was further extracted with ethyl acetate (50 mL) and combined with the organic layer. The organic layer was washed with saturated brine (100 mL), followed by reduced pressure concentration to obtain an intermediate 3c, which was all used in the sequential reaction. HRMS(ESI⁺, M+H): calc. 533.2070, found 533.2074.

The intermediate 3c was mixed with 85% phosphoric acid (3 mL) and water (1 mL), and the mixture was heated and stirred for 8 hr under reflux. After the mixture was cooled down to room temperature, the mixture was added into water (100 mL), and neutralized with 45% NaOH aqueous solution to pH 6.5. Isopropanol (100 mL), toluene (100 mL) and NaCl (10 g) was added into the mixture, followed by extraction. The water layer was further extracted with isopropanol (50 mL) and toluene (50 mL) and combined with the organic layer. The organic layer was washed with saturated brine (100 mL), followed by reduced pressure concentration and crystallized with toluene/methanol to obtain the compound 2d (0.12 g). The final yield after the aforesaid four steps was 33.5%.

¹H NMR (DMSO-d⁶, 400 MHz): 1.45 (s, 9H), 1.89 (quint, J=6.3 Hz, 2H), 2.94 (s, 2H), 3.59 (t, J=6.3 Hz, 2H), 3.65 (s, 2H), 4.06 (t, J=6.3 Hz, 2H), 7.00 (d, J=2.8 Hz, 1H), 7.49 (t, J=2.8 Hz, 1H), 7.98 (d, J=8.8 Hz, H), 8.35 (d, J=8.8 Hz, 1H), 8.72 (d, J=2.8 Hz, 1H); ¹³C NMR (DMSO-d⁶, 100 MHz): 29.2, 32.1, 34.7, 35.3, 55.3, 57.3, 65.3, 104.9, 117.3, 119.9, 120.5, 124.8, 126.4, 137.9, 140.6, 141.9, 143.0, 144.6, 151.1; HRMS(ESI, M+H): calc. 449.1859, found 449.1859.

### Preparation Example 2: Synthesis of the Raw Compound 1b Synthesis of the Precursor Compound 4

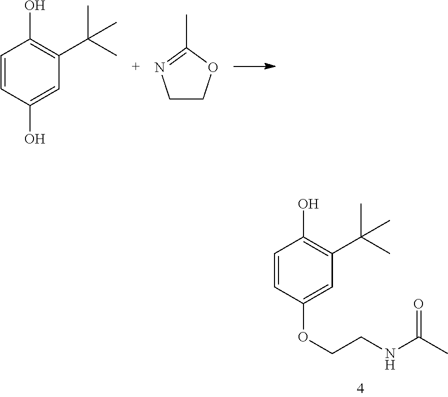

To a flask (250 mL), tert-butylhydroquinone (33.24 g) and 2-methyl-2-oxazoline (20.42 g) were added, and the mixture was heated to 160° C. and stirred for 16 hr. After the mixture was cooled down to room temperature, the mixture was extracted with toluene (100 mL), ethyl acetate (200 mL) and water (100 mL). The water layer was removed, and the organic layer was further washed with saturated brine (100 mL). The organic layer was dried and concentrated. After reduced pressure distillation at 0.25 mbar, and the distillate obtained in the region with the boiling point between 215° C. and 220° C. contained the compound 4 (30.31 g, 42.2%).

¹H NMR (Acetone-d⁶, 400 MHz): 1.38 (s, 9H), 1.93 (s, 3H), 3.53 (q, J=5.6 Hz, 2H), 3.94 (t, J=5.6 Hz, 2H), 6.58 (dd, J=2.8, 8.8 Hz, 1H), 6.74 (d, J=8.8 Hz, 1H), 6.78 (d, J=2.8 Hz, 1H), 7.51 (br s, 1H), 8.18 (br s, 1H); ¹³C NMR (Acetone-d⁶, 100 MHz): 22.8, 29.8, 35.2, 39.8, 67.8, 112.2, 115.0, 117.2, 137.6, 150.8, 152.7, 170.8.

### Synthesis of the Raw Compound 1b

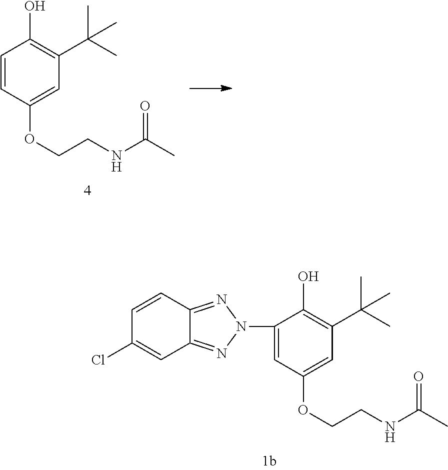 [SMILES: CC(=O)NCCOc1ccc(O)c(C(C)(C)C)c1 ; CC(=O)NCCOc1cc(-n2nc3ccc(Cl)cc3n2)c(O)c(C(C)(C)C)c1]

4-Chloro-2-nitroaniline (16 g), a dispersant (0.05 g), concentrated HCl (50 ml) and ice (100 g) were mixed and stirred for 1 hr. After the mixture was cooled down to 5° C., a sodium nitrite aqueous solution (6.8 g sodium nitride dissolved in 50 mL water) was slowly added. After adding the sodium nitrite aqueous solution, the mixture was stirred for further 0.5 hr, followed by adding ammonium sulfamate (0.3 g) and stirring for 0.5 hr. After adding diatomaceous earth (0.3 g) and stirring for 0.5 hr, the mixture was filtered to obtain an azo solution, and the azo solution was placed at low temperature (<5° C.) for the sequential reaction.

The compound 4 (18.71 g), a dispersant (0.1 g), 45% NaOH aqueous solution (10 g), ice (100 g) and ethanol (500 mL) were mixed and stirred for 1 hr. After cooling down to 5° C., the prepared azo solution was slowly added into the mixture dropwise, and 45% NaOH aqueous solution was added when needed to control the pH value between 8 and 10.5. After adding the azo solution, the temperature of the mixture was back to room temperature, and the mixture was stirred for 16 hr. Then, 45% NaOH aqueous solution (66 g) as added, and the mixture was heated and stirred for 0.5 hr under reflux. Sodium dithionite (16 g) was added and stirred for 1 hr, and then sodium dithionite (16 g) was further added and stirred for 1 hr, and then sodium dithionite (32 g) and 45% NaOH aqueous solution (16 g) was further added and stirred for further 1 hr. After the mixture was back to room temperature, the mixture was filtered and the solids was collected to obtain the compound 1b (13.3 g, 44.3%).

¹H NMR (DMSO-t, 400 MHz): 1.45 (s, 9H), 1.84 (s, 3H), 3.43 (q, J=5.2 Hz, 2H), 4.02 (t, J=5.2 Hz, 2H), 7.00 (s, 1H), 7.53 (s, 1H), 7.59 (d, J=9.0 Hz, 1H), 8.12 (s, 1H), 8.14 (d, J=9.0 Hz, H), 8.25 (s, 1H), 10.64 (br s, 1H); ¹³C NMR (DMSO-d⁶, 100 MHz): 22.5, 29.2, 35.3, 38.3, 67.1, 104.6, 116.9, 119.7, 126.0, 129.0, 132.4, 140.4, 141.4, 142.8, 143.2, 150.7, 169.5.

### Example 5: Synthesis of Compound 2e (i.e. the Compound (I-5) of the Present Disclosure)

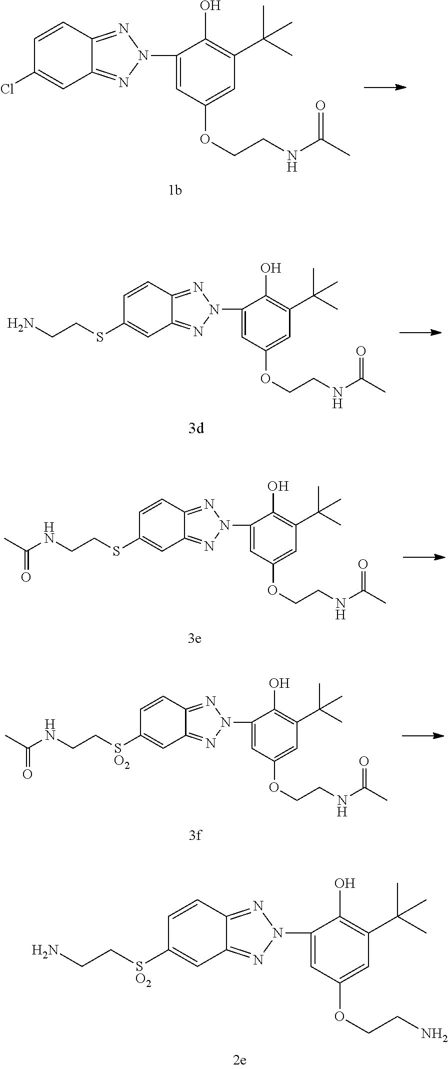

To a flask (25 mL), the compound 1b (1.25 g), potassium carbonate (4.0 g), 2-aminoethanethiol (1.2 g) and N-methyl-2-pyrrolidone (10 mL) were added, and the mixture was heated to 100° C. and stirred for 12 hr. After cooling down to room temperature, the mixture was added into water (300 mL) and acidified with 1 N HCl aqueous solution to pH 6. The mixture was extracted with isopropanol (25 mL), toluene (75 mL) and NaCl (20 g). The water layer was extracted with isopropanol (25 mL) and toluene (75 mL) and then combined with the organic layer. The organic layer was washed with saturated brine (100 mL), concentrated under reduced pressure, and purified with column chromatography to obtain an intermediate 3d, which was all used in the sequential reaction.

The intermediate 3d was mixed with ethyl acetate (50 mL), tert-butanol (9 mL), acetic anhydride (12 mL) and triethylamine (9 mL), the mixture was heated to 70° C. and stirred. After the intermediate 3d was completely dissolved, the mixture was cooled down to room temperature. The mixture was extracted with water (100 mL) and ethyl acetate (100 mL). The water layer was further extracted with ethyl acetate (50 mL) and then combined with the organic layer. The organic layer was washed with saturated brine (50 mL) and concentrated under reduced pressure to obtain an intermediate 3e, which was all used in the sequential reaction.

The intermediated 3e was mixed with Na₂WO₄.2H₂O (0.1 g), 30% H₂O₂ aqueous solution (15 mL), water (5 mL) and isopropanol (50 mL), and the mixture was stirred at room temperature for 16 hr. The mixture was extracted with water (100 mL) and ethyl acetate (100 mL). The water layer was further extracted with ethyl acetate (50 mL) and then combined with the organic layer. The organic layer was washed with saturated brine (100 mL) and concentrated under reduced pressure to obtain an intermediate 3f, which was all used in the sequential reaction.

The intermediated 3f was mixed with 85% phosphoric acid (12 mL) and water (3 mL), and the mixture was heated under reflux for 8 hr. After cooling down to room temperature, the mixture was added into water (200 mL), and neutralized with 45% NaOH aqueous solution to pH 6.5. The mixture was filtered to collect solids, and the solids were washed with water (50 mL). The filter cake was recrystallized with isopropanol to obtain the compound 2e (0.13 g). The total yield after four steps was 9.7%.

¹H NMR (DMSO-d⁶, 400 MHz): 1.46 (s, 9H), 2.92 (t, J=7.2 Hz, 2H), 3.18 (t, J=4.8 Hz, 2H), 3.64 (t, J=7.2 Hz, 2H), 4.22 (t, J=4.8 Hz, 2H), 7.11 (d, J=2.8 Hz, 1H), 7.55 (d, J=2.8 Hz, 1H), 7.98 (d, J=2.8 Hz, 1H), 8.00 (dd, J=1.2, 9.2 Hz, 1H), 8.38 (d, J=9.2 Hz, 1H), 8.74 (d, J=1.2 Hz, 1H); ¹³C NMR (DMSO-d⁶, 100 MHz): 29.2, 35.2, 35.3, 38.6, 56.0, 65.7, 105.8, 117.7, 119.9, 120.5, 124.8, 126.5, 138.1, 140.7, 142.0, 143.6, 144.7, 150.3; HRMS(ESI⁺, M+H): calc. 434.1862, found 434.1863.

### Example 6: Synthesis of Compound 2f (i.e. the Compound (I-6) of the Present Disclosure)

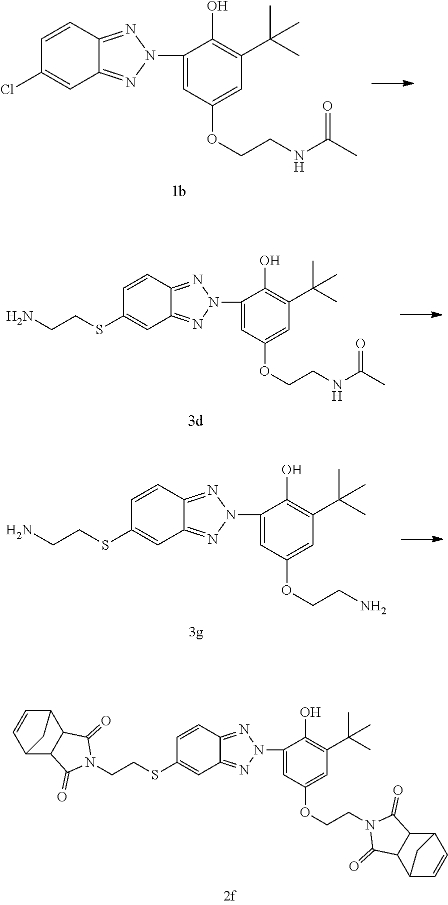 [SMILES: CC(=O)NCCOc1cc(-n2nc3ccc(Cl)cc3n2)c(O)c(C(C)(C)C)c1 ; CC(=O)NCCOc1cc(-n2nc3ccc(SCCN)cc3n2)c(O)c(C(C)(C)C)c1 ; CC(C)(C)c1cc(OCCN)cc(-n2nc3ccc(SCCN)cc3n2)c1O ; CC(C)(C)c1cc(OCCN2C(=O)C3C4C=CC(C4)C3C2=O)cc(-n2nc3ccc(SCCN4C(=O)C5C6C=CC(C6)C5C4=O)cc3n2)c1O]

To a flask (25 mL), the compound 1b (2.88 g), potassium carbonate (3.0 g), 2-aminoethanethiol (3.0 g) and N-methyl-2-pyrrolidone (10 mL) were added, the mixture was heated to 100° C. and stirred for 12 hr. After cooling down to room temperature, the mixture was added into water (300 mL) and acidified with 1 N HCl aqueous solution to pH 5. The mixture was filtered, and the collected solids were washed with water (100 mL) to obtain an intermediate 3d (1.5 g), which was all used in the sequential reaction.

The intermediate 3d was mixed with 85% phosphoric acid (12 mL) and water (3 mL), and the mixture was stirred and heated under reflux for 8 hr. After cooling down to room temperature, the mixture was added into water (200 mL), and neutralized with 45% NaOH aqueous solution to pH 6.5. The mixture was filtered, and the collected solids were washed with water (50 mL). The filter cake was recrystallized with isopropanol to obtain an intermediate 3g (5 g).

The intermediate 3g (0.15 g) was added into 5-norbornene-2,3-dicarboxylic anhydride (0.15 g) and toluene (20 mL), and the mixture was stirred under reflux for 16 hr. After cooling down to room temperature, the mixture was concentrated under reduced pressure and purified with column chromatography to obtain the compound 2f (0.1 g). The total yield of the three steps was 15.1%.

¹H NMR (CDCl₃, 400 MHz): 1.47 (dt, J=8.8, 1.6 Hz, 1H), 1.48 (s, 9H), 1.54 (dt, J=8.8, 1.6 Hz, 1H), 1.73 (dt, J=8.8, 1.6 Hz, 1H), 1.74 (dt, J=8.8, 1.6 Hz, 1H), 3.10 (t, J=7.6 Hz, 2H), 3.24-3.27 (m, 1H), 3.29-3.32 (m, 1H), 3.38-3.43 (m, 4H), 3.66 (t, J=7.6 Hz, 2H), 3.81 (t, J=5.6 Hz, 2H), 4.10 (t, J=5.6 Hz, 2H), 6.08 (t, J=1.6 Hz, 2H), 6.14 (t, J=1.6 Hz, 2H), 6.95 (d, J=3.0 Hz, 1H), 7.40 (dd, J=8.8, 3.2 Hz, 1H), 7.72 (d, J=3.0 Hz, 1H), 7.83 (d, J=8.8 Hz, 1H), 7.90 (d, J=3.2 Hz, 1H), 11.46 (s, 1H); ¹³C NMR (CDCl₃, 100 MHz): 29.5, 30.3, 35.7, 37.3, 37.8, 45.4, 45.9, 52.2, 52.3, 64.9, 103.2, 115.3, 117.0, 118.0, 125.2, 129.5, 134.4, 134.6, 135.7, 140.9, 141.5, 143.3, 143.6, 150.7, 177.5, 177.6; HRMS(ESI⁺, M+H): calc. 694.2699, found 694.2775.

### Preparation Example 3: Synthesis of the Raw Compound 1c

Synthesis of the Raw Compound 1c′

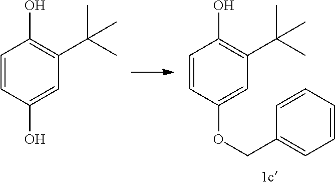 [SMILES: CC(C)(C)c1cc(O)ccc1O ; CC(C)(C)c1cc(OCc2ccccc2)ccc1O]

The preparation process can be referred to that for the compound 1a′. Herein, the starting materials were tert-butylhydroquinone (856.8 g) and benzyl bromide (960.0 g), and light yellow liquid (670.0 g) was obtained.

### Synthesis of the Raw Compound 1c

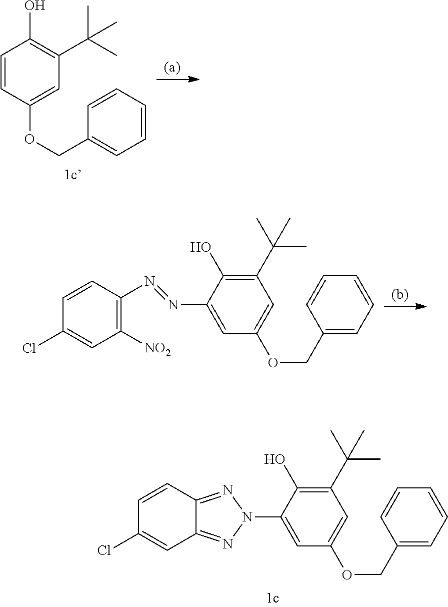 [SMILES: CC(C)(C)c1cc(OCc2ccccc2)ccc1O ; CC(C)(C)c1cc(OCc2ccccc2)cc(/N=N/c2ccc(Cl)cc2[N+](=O)[O-])c1O ; CC(C)(C)c1cc(OCc2ccccc2)cc(-n2nc3ccc(Cl)cc3n2)c1O]

The preparation process can be referred to that for the compound 1a. Herein, the starting material was the raw compound 1c′ (450.0 g), and yellow solids (138.0 g, m.p. 130.7° C.) were obtained.

¹H NMR (400 MHz, CDCl₃): δ 11.36 (1H), 7.80-8.00 (3H), 7.49 (2H), 7.30-7.45 (4H), 7.10 (1H), 5.11 (2H), 1.48 (9H); HRMS ESI [M−H]: calc. 406.1322, found 406.1324.

### Example 7: Synthesis of Compound 2g (i.e. the Compound (1-7) of the Present Disclosure)

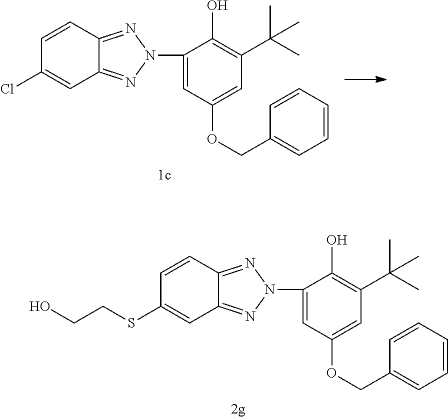 [SMILES: CC(C)(C)c1cc(OCc2ccccc2)cc(-n2nc3ccc(Cl)cc3n2)c1O ; CC(C)(C)c1cc(OCc2ccccc2)cc(-n2nc3ccc(SCCO)cc3n2)c1O]

To a flask (20 mL), the compound 1c (2.0 g), KOH (0.6 g), potassium carbonate (10.0 g), 2-mercaptoethanol (3 mL) and N-methylpyrrolidone (20 mL) were added. The mixture was heated to 140° C. and stirred for 8 hr. After cooling down to room temperature, the mixture was acidified with 1 N HCl aqueous solution to pH 5. The mixture was extracted with toluene (100 mL), ethyl acetate (50 mL) and water (100 mL). The water layer was removed, and the organic layer was dried with MgSO₄ to remove water. After reduced pressure concentration and purification with column chromatography, the compound 2g (0.8 g, 36.3%) was obtained. ¹H NMR (CDCl₃, 400 MHz): 1.48 (s, 9H), 3.25 (t, J=6.0 Hz, 2H), 3.87 (t, J=6.0 Hz, 2H), 5.12 (s, 2H), 7.09 (d, J=3.2 Hz, 1H), 7.29-7.55 (m, 6H), 7.77-7.93 (m, 3H), 11.44 (s, 1H); ¹³C NMR (CDCl₃, 100 MHz): 29.6, 35.8, 36.9, 60.5, 70.9, 103.4, 115.9, 117.6, 118.1, 125.3, 127.9, 128.2, 128.8, 129.9, 136.0, 137.0, 141.1, 141.6, 143.3, 143.7, 151.3; HRMS(ESI⁺, M+H): calc. 450.1851, found 450.1863.

### Example 8: Synthesis of Compound 2h (i.e. the Compound (I-8) of the Present Disclosure)

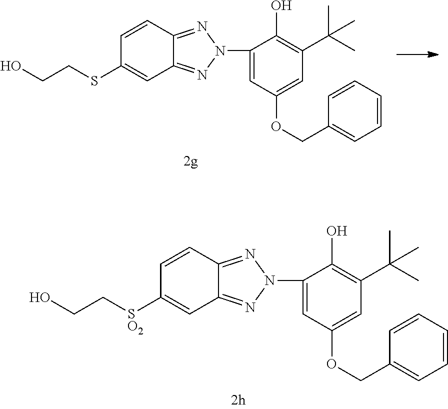 [SMILES: CC(C)(C)c1cc(OCc2ccccc2)cc(-n2nc3ccc(SCCO)cc3n2)c1O ; CC(C)(C)c1cc(OCc2ccccc2)cc(-n2nc3ccc(S(=O)(=O)CCO)cc3n2)c1O]

To a flask (250 mL), the compound 2g (0.4 g), Na₂WO₄ 2H₂O (0.1 g), 30% H₂O₂ aqueous solution (15 mL), isopropanol (50 mL) and water (5 mL) were added and stirred for 16 hr. The mixture was extracted with toluene (100 mL) and NaCl (10 g). The water layer was further extracted with toluene (100 mL) and combined with the organic layer. The organic layer was washed with saturated brine (100 mL), dried with MgSO₄, concentrated under reduced pressure and purified with column chromatography to obtain the compound 2h (0.28 g, 65.3%).

¹H NMR (CDCl₃, 400 MHz): 1.49 (s, 9H), 2.66 (t, J=2.4 Hz, 1H), 3.47 (dt, J=2.4, 5.2 Hz, 2H), 4.09 (t, J=5.2 Hz, 2H), 5.14 (s, 2H), 7.16 (d, J=2.8 Hz, 1H), 7.30-7.47 (m, 3H), 7.50 (d, J=7.2 Hz, 2H), 7.89-7.99 (m, 2H), 8.14 (d. J=9.2 Hz, 1H), 8.68 (s, 1H), 11.25 (s, 1H); ¹³C NMR (CDCl₃, 100 MHz): 29.5, 35.9, 56.6, 58.6, 71.0, 103.5, 119.0, 119.6, 120.9, 125.0, 127.9, 128.3, 128.8, 136.8, 138.3, 141.6, 141.7, 144.3, 144.5, 151.4; HRMS(ESI⁺, M+H): calc. 482.1750, found 482.1764.

### Example 9: Synthesis of Compound 2i (i.e. the Compound (I-9) of the Present Disclosure)

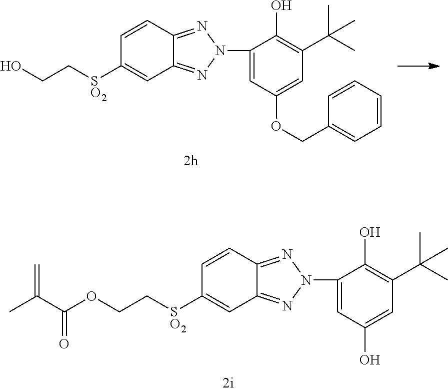 [SMILES: CC(C)(C)c1cc(OCc2ccccc2)cc(-n2nc3ccc(S(=O)(=O)CCO)cc3n2)c1O ; C=C(C)C(=O)OCCS(=O)(=O)c1ccc2nn(-c3cc(O)cc(C(C)(C)C)c3O)nc2c1]

To a flask (250 mL), the compound 2h (0.2 g), hydroquinone (0.1 g), p-toluenesulfonic acid (0.1 g), methacrylic acid (0.5 mL) and toluene (75 mL) were added, and the mixture was heated and stirred under reflux for 16 hr. After cooling down to room temperature, the mixture was extracted with toluene (25 mL) and saturated NaHCO₃ aqueous solution (100 mL). The water layer was further extracted with toluene (50 mL) and combined with the organic layer. The organic layer was washed with saturated brine (100 mL), dried with MgSO₄, concentrated under reduced pressure, purified with column chromatography and recrystallized with methanol/toluene to obtain the compound 2i (0.15 g, 78.6%).

¹H NMR (CDCl₃, 400 MHz): 1.49 (s, 9H), 1.72 (d, J=1.6 Hz, 3H), 1.97 (d, J=1.2 Hz, 3H), 3.64 (t, J=6.0 Hz, 2H), 4.57 (t, J=6.0 Hz, 2H), 5.38 (d, J=1.6 Hz, 1H), 5.76 (s, 1H), 7.02 (d, J=2.8 Hz, 1H), 7.78 (d, J=2.8 Hz, 1H), 7.92 (dd, J=1.2 Hz, 9.2 Hz, 1H), 8.08 (d, J=9.2 Hz, 1H), 8.64 (d, J=1.2 Hz, 1H), 11.13 (s, 1H); ¹³C NMR (CDCl₃, 100 MHz): 18.1, 29.5, 35.9, 55.4, 58.0, 105.6, 117.9, 119.5, 121.1, 125.1, 126.8, 135.3, 138.6, 141.6, 141.7, 143.8, 144.4, 148.1, 166.7; HRMS(ESI⁺, M+H): calc. 460.1542, found 460.1549.

### Preparation Example 4: Synthesis of the Raw Compound 1d

### Synthesis of the Raw Compound 1d′

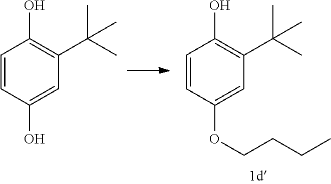 [SMILES: CC(C)(C)c1cc(O)ccc1O ; CCCCOc1ccc(O)c(C(C)(C)C)c1]

The preparation process can be referred to that for the compound 1a′. Herein, the starting materials were tert-butylhydroquinone (856.8 g) and butyl bromide (706.3 g), and light yellow liquid (590.1 g) was obtained.

### Synthesis of the Raw Compound 1 d

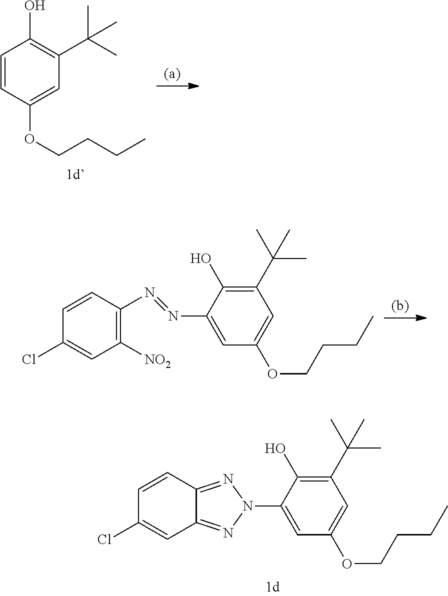 [SMILES: CCCCOc1ccc(O)c(C(C)(C)C)c1 ; CCCCOc1cc(/N=N/c2ccc(Cl)cc2[N+](=O)[O-])c(O)c(C(C)(C)C)c1 ; CCCCOc1cc(-n2nc3ccc(Cl)cc3n2)c(O)c(C(C)(C)C)c1]

The preparation process can be referred to that for the compound 1a. Herein, the starting material was the raw compound 1d′ (450.0 g), and yellow solids (159.6 g, m.p. 101.6° C.) were obtained.

HRMS ESI [M−H]⁻: calc. 372.1479, found 372.1477.

¹H NMR (400 MHz, CDCl₃): δ 11.31 (1H), 7.91 (1H), 7.85 (1H), 7.75 (1H), 7.41 (1H), 7.02 (1H), 4.03 (2H), 1.81 (2H), 1.54 (2H), 1.49 (9H), 1.00 (3H)

### Example 10: Synthesis of Compound 2j (i.e. the Compound (I-10) of the Present Disclosure)

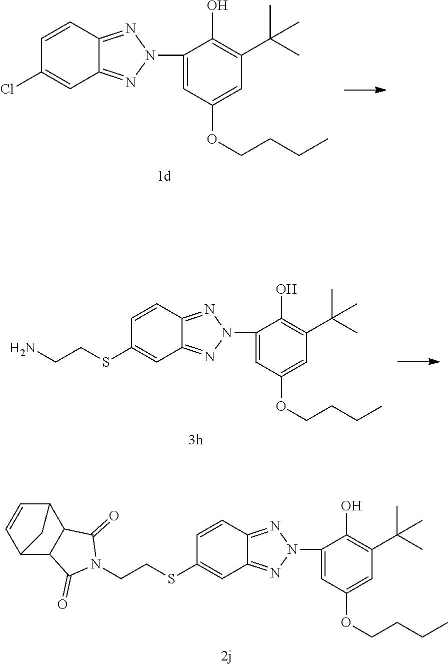 [SMILES: CCCCOc1cc(-n2nc3ccc(Cl)cc3n2)c(O)c(C(C)(C)C)c1 ; CCCCOc1cc(-n2nc3ccc(SCCN)cc3n2)c(O)c(C(C)(C)C)c1 ; CCCCOc1cc(-n2nc3ccc(SCCN4C(=O)C5C6C=CC(C6)C5C4=O)cc3n2)c(O)c(C(C)(C)C)c1]

To a flask (25 mL), the compound 1d (1.0 g), potassium carbonate (1.6 g), 2-aminoethanethiol (1.0 g), and N-methylpyrrolidone (10 mL) were added. The mixture was heated to 120° C. and stirred for 16 hr. After cooling down to room temperature, the mixture was added into water (300 mL) and acidified with 1N HCl aqueous solution to pH 5. The mixture was extracted with toluene (200 mL) and NaCl (10 g). The water layer was extracted with toluene (100 mL) and combined with the organic layer. The organic layer was washed with saturated brine (100 mL), concentrated under reduced pressure and purified with column chromatography to obtain an intermediate 3h (0.3 g), which was all used in the sequential reaction. HRMS(ESI⁺, M+H): calc. 415.2168, found 415.2164.

The intermediate 3h was mixed with 5-norbornene-2,3-dicarboxylic anhydride (1.0 g), dimethylformamide (20 mL) and toluene (20 mL), and the mixture was stirred under reflux for 16 hr. After cooling down to room temperature, the mixture was extracted with toluene (200 mL), NaCl (10 g) and saturated ammonium chloride aqueous solution (100 mL). The water layer was further extracted with ethyl acetate (100 mL) and combined with the organic layer. The organic layer was washed with saturated brine (100 mL), concentrated under reduced pressure and purified with column chromatography to obtain the compound 2j (0.21 g). The total yield of the two steps was 14.0%.

¹H NMR (CDCl₃, 400 MHz): 1.03 (t, J=7.6 Hz, 3H), 1.45-1.65 (m, 12H), 1.70-1.90 (m, 3H), 3.08 (t, J=7.2 Hz, 2H), 3.20-3.30 (m, 2H), 3.40-3.47 (m, 2H), 3.65 (t, J=7.2 Hz, 2H), 4.04 (t, J=6.4 Hz, 2H), 6.14 (s, 2H), 7.01 (d, J=2.8 Hz, 1H), 7.39 (dd, J=9.2, 1.4 Hz, 1H), 7.76 (d, J=2.8 Hz, 1H), 7.83 (d, J=9.2 Hz, 1H), 7.90 (d, J=1.4 Hz, 1H), 11.43 (s, 1H); ¹³C NMR (CDCl₃, 00 MHz): 14.0, 19.4, 29.5, 30.3, 31.6, 35.7, 37.4, 45.1, 45.9, 52.3, 68.5, 102.8, 115.4, 117.2, 118.0, 125.2, 129.5, 134.6, 135.6, 140.8, 141.5, 143.3, 143.4, 151.5, 177.5; HRMS(ESI⁺, M+H): calc. 561.2536, found 561.2608.

### Example 11: Synthesis of Compound 2k (i.e. the Compound (I-11) of the Present Disclosure)

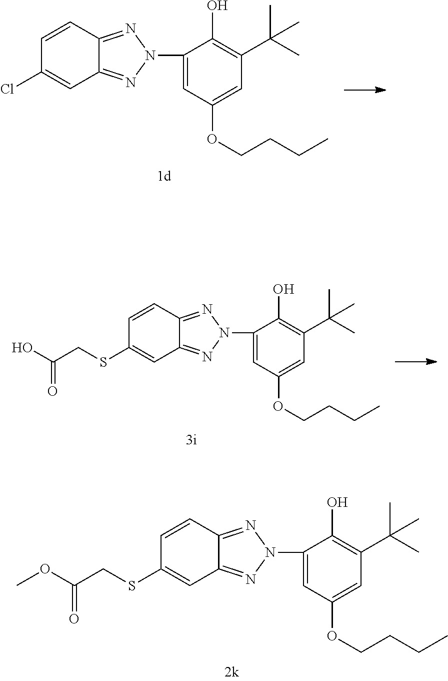

To a flask (25 mL), the compound 1d (1.0 g), potassium carbonate (1.6 g), mercaptoacetic acid (1.0 mL) and N-methylpyrrolidone (10 mL) were added, and the mixture was heated to 140° C. and stirred for 16 hr. Then, potassium carbonate (1.6 g) was further added therein and the mixture was further heated for 16 hr. After cooling down to room temperature, the mixture was added into water (300 mL) and acidified with 1N HCl aqueous solution to pH 5. The mixture was extracted with isopropanol (25 mL), ethyl acetate (75 mL) and NaCl (10 g). The water layer was extracted with isopropanol (25 mL) and ethyl acetate (75 mL) and combined with the organic layer. The organic layer was washed with saturated brine (100 mL) and concentrated under reduced pressure to obtain an intermediate 3i, which was all used in the sequential reaction.

The intermediate 3i was mixed with p-toluenesulfonic acid (0.5 g), toluene (20 mL) and methanol (100 mL), and the mixture was stirred under reflux for 16 hr. After cooling down to room temperature, the mixture was extracted with toluene (200 mL), NaCl (10 g) and saturated NaHCO₃ aqueous solution (100 mL). The water layer was further extracted with ethyl acetate (100 mL) and combined with the organic layer. The organic layer was washed with saturated brine (100 mL), concentrated under reduced pressure and purified with column chromatography to obtain the compound 2k (0.34 g). The total yield of the two steps was 28.7%.

¹H NMR (CDCl₃, 400 MHz): 1.01 (t, J=7.2 Hz, 3H), 1.45-1.65 (m, 11H), 1.75-1.85 (m, 2H), 3.76 (s, 3H), 3.78 (s, 2H), 3.40-3.47 (m, 2H), 3.65 (t, J=7.2 Hz, 2H), 4.03 (t, J=6.4 Hz, 2H), 7.01 (d, J=3.2 Hz, 1H), 7.44 (dd, J=8.8, 1.4 Hz, 1H), 7.75 (d, J=3.2 Hz, 1H), 7.84 (d, J=8.8 Hz, 1H), 7.87 (d, J=1.4 Hz, 1H), 11.43 (s, 1H); ¹³C NMR (CDCl₃, 100 MHz): 14.1, 19.5, 29.6, 31.6, 35.8, 36.2, 52.9, 68.5, 102.8, 116.3, 117.4, 118.1, 125.3, 129.6, 135.6, 141.0, 141.7, 143.3, 143.4, 151.6, 169.8; HRMS(ESI⁺, M+H): calc. 444.1957, found 444.1957.

### Example 11: Synthesis of Compound 2l (i.e. the Compound (I-12) of the Present Disclosure)

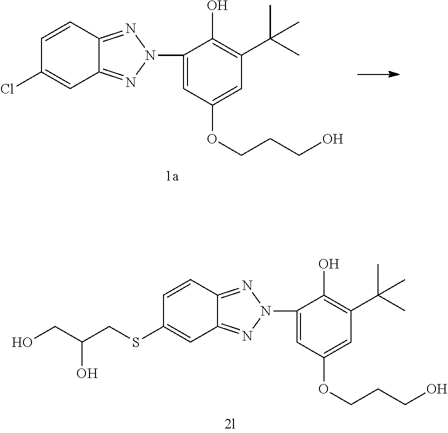 [SMILES: CC(C)(C)c1cc(OCCCO)cc(-n2nc3ccc(Cl)cc3n2)c1O ; CC(C)(C)c1cc(OCCCO)cc(-n2nc3ccc(SCC(O)CO)cc3n2)c1O]

To a flask (20 mL), the compound 1a (5.0 g), potassium carbonate (5.0 g), monothioglycerol (3.0 mL) and N-methylpyrrolidone (15 mL) were added, and the mixture was heated to 140° C. and stirred for 4 hr. After cooling down to room temperature, the mixture was added into water (200 mL) and acidified with 1N HCl aqueous solution to pH 4.5. The mixture was filtered, and the collected solids were washed with water (100 mL). The filter cake was added into isopropanol (100 mL) and heated to 60° C. to dissolve the filter cake. Then, heptane (300 mL) was added into the mixture, and the temperature of the mixture was slowly cooled down. Then, water (200 mL) was added into the mixture and the temperature of the mixture was cooled down to 5° C. The precipitated solids were collected and further purified with column chromatography to obtain the compound 2l (4.53 g, 76.0%).

¹H NMR (CDCl₃, 400 MHz): 1.45 (s, 9H), 1.91 (quint, J=6.4 Hz, 2H), 3.06 (dd, J=7.0, 13.2 Hz, 1H), 3.20 (dd, J=4.4, 13.2 Hz, 1H), 3.40-3.55 (m, 2H), 3.60 (q, J=5.8 Hz, 2H), 3.70-3.82 (m, 1H), 4.07 (t, J=6.6 Hz, 2H), 4.60 (t, J=5.2 Hz, 1H), 4.80 (t, J=5.8 Hz, 1H), 5.15 (d, J=5.2 Hz, 1H), 6.94 (d, J=3.0 Hz, 1H), 7.48 (dd, J=1.2, 9.0 Hz, 1H), 7.59 (d, J=3.0 Hz, 1H), 7.91 (d, J=1.2 Hz, 1H), 7.98 (d, J=9.0 Hz, 1H), 11.05 (s, 1H); ¹³C NMR (CDCl3, 100 MHz): 29.2, 32.2, 35.3, 36.1, 57.4, 64.7, 65.3, 70.1, 103.3, 112.3, 116.3, 117.8, 125.5, 129.0, 138.6, 140.2, 140.9, 142.3, 143.3, 151.0; HRMS(ESI⁺, M+H): calc. 448.1906, found 418.1913.

### Comparative Example 1

The commercial available product, Eversorb® 82, is represented by the following formula (II).

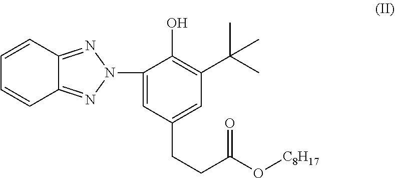

### Comparative Example 2: Synthesis of Compound 6

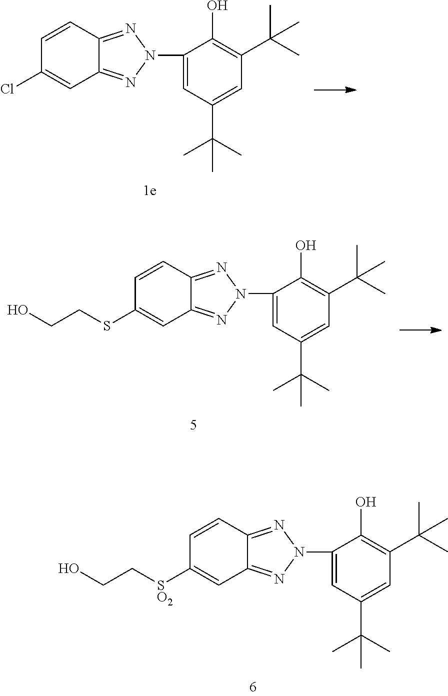 [SMILES: CC(C)(C)c1cc(-n2nc3ccc(Cl)cc3n2)c(O)c(C(C)(C)C)c1 ; CC(C)(C)c1cc(-n2nc3ccc(SCCO)cc3n2)c(O)c(C(C)(C)C)c1 ; CC(C)(C)c1cc(-n2nc3ccc(S(=O)(=O)CCO)cc3n2)c(O)c(C(C)(C)C)c1]

To a flask (20 mL), the compound 1e (commercial available product, Eversorb® 75) (4.0 g), KOH (1.3 g), KI (0.05 g), 2-mercaptoethanol (0.9 mL) and N-methylpyrrolidone (10 mL) were added, and the mixture was heated to 100° C. and stirred for 6 hr. After cooling down to room temperature, the mixture was acidified with 1N HCl aqueous solution to pH 5. The mixture was extracted with toluene (100 mL) and water (100 mL). The water layer was removed, and the organic layer was dried with MgSO₄ to remove water. After reduced pressure concentration and purification with column chromatography, an intermediate 5 (1.94 g, 43%) was obtained. HRMS(ESI⁺, M+H): calc. 400.2059, found 400.2059.

To a flask (250 mL), the intermediate 5 (0.9 g), Na₂WO₄.2H₂O (0.15 g), toluene (25 mL), isopropanol (50 mL) and 30% H₂O₂ aqueous solution (15 mL) were added, and the mixture was stirred for 16 hr. The mixture was extracted with toluene (85 mL) and water (50 mL). The water layer was further extracted with toluene (75 mL) and combined with organic layer. The organic layer was washed with saturated brine (50 mL), dried with MgSO₄, and concentrated under reduced pressure to obtain the compound 6 (0.89 g, 91.6%).

¹H NMR (CDCl₃, 400 MHz): 1.40 (s, 9H), 1.52 (s, 9H), 2.45 (br s, 1H), 3.47 (t, J=5.4 Hz, 2H), 4.08 (t, J=5.4 Hz, 2H), 7.50 (d, J=2.4 Hz, 1H), 7.94 (dd, J=8.8, 1.4 Hz, 1H), 8.14 (d, J=8.8 Hz, 1H), 8.31 (d, J=8.8 Hz, 1H), 8.69 (d, J=1.4 Hz, 1H), 11.39 (s, 1H); ¹³C NMR (CDCl₃, 100 MHz): δ 29.7, 31.6, 34.8, 35.9, 56.5, 58.6, 116.6, 119.6, 120.9, 124.8, 125.0, 126.7, 138.1, 139.2, 141.7, 142.4, 144.5, 147.3; HRMS (ESI⁺, M+H): calc. 432.1957, found 432.1957.

### Comparative Example 3

The commercial available product, Eversorb® 78, is represented by the following formula (III).

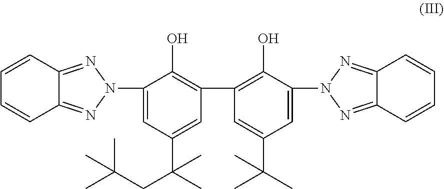 [SMILES: CC(C)(C)CC(C)(C)c1cc(-c2cc(C(C)(C)C)cc(-n3nc4ccccc4n3)c2O)c(O)c(-n2nc3ccccc3n2)c1]

### Comparative Example 4: Synthesis of Compound 7

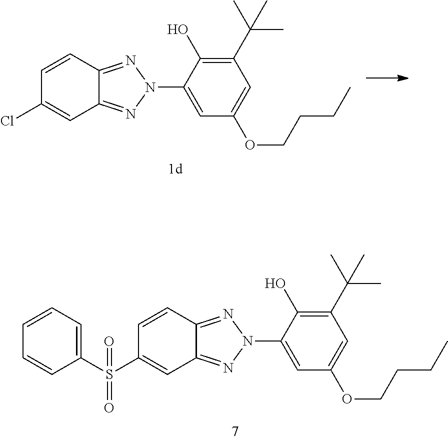 [SMILES: CCCCOc1cc(-n2nc3ccc(Cl)cc3n2)c(O)c(C(C)(C)C)c1 ; CCCCOc1cc(-n2nc3ccc(S(=O)(=O)c4ccccc4)cc3n2)c(O)c(C(C)(C)C)c1]

The compound 1d (40 g) was dissolved in N-methylpyrrolidone (10.5 g). At 90° C., 45% KOH aqueous solution (4.43 g) was slowly added into the mixture, and then thiophenol (2.04 g) was added dropwise into the mixture. After addition, the mixture was heated to 170° C. to remove water for 24 hr. Then, the mixture was cooled down to 100° C., extracted with xylene (75 mL) and washed with water (75 mL). The water layer was acidified with 15% HCl, followed by removing the water layer. The organic layer was dried with anhydrous sodium sulfate and concentrated. After placing a period of time, yellow solids were precipitated.

Next, the obtained yellow solids were dissolved in xylene (4.28 g), and sodium tungstate (0.038 g) and 90% formic acid (0.85 g) were added into the mixture. The mixture was heated to 50° C., and 30% H₂O₂ (2.54 g) was added dropwise into the mixture. The temperature of the mixture was less than 85° C. during the addition. The reaction was traced with thin layer chromatography. Then, xylene and water were added into the mixture. After extraction, drying and concentration, the product was recrystallized in methanol to obtain yellow solids, compound 7 (35.4 g, m.p. 148.3° C.).

HRMS ESI [M+H]⁺: calc. 480.1957, found 480.1964.

¹H NMR (400 MHz, CDCl₃): δ 11.21 (l H), 8.71 (1H), 7.95-8.05 (3H), 7.90 (1H), 7.77 (1H), 7.40-7.70 (3H), 7.06 (H), 4.03 (2H), 1.81 (2H), 1.54 (2H), 1.48 (9H), 1.00 (3H)

### Test Example 1: Comparison of UV Absorption Spectra

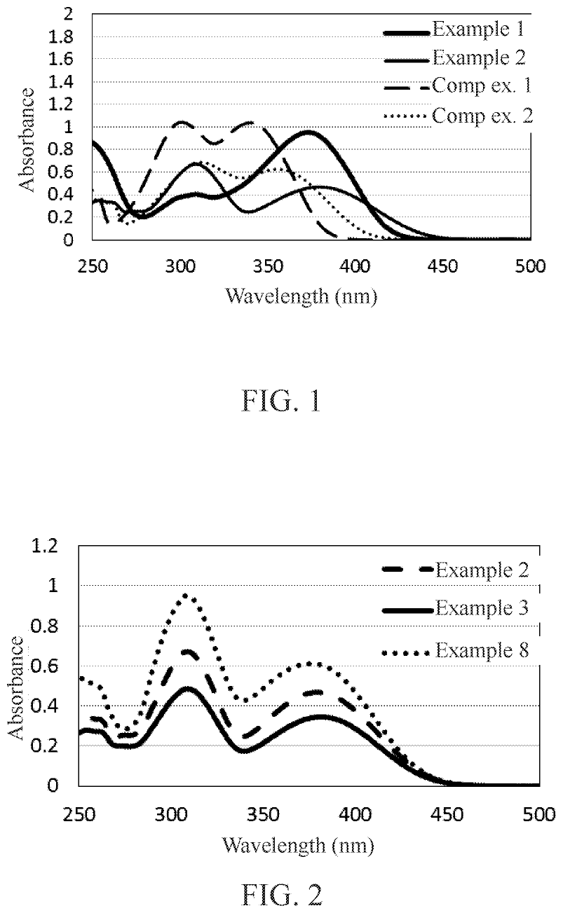

The UV absorbers of Examples 1, 2, 3 and 8 and Comparative examples 1 and 2 were formulated into 20 ppm solutions with methanol/THF (methanol:THF=90:10). An UV/Visible spectrometer (UV-2600; Shimadzu Instruments Co., Ltd.) was used to measure the absorption of the UV absorbers. The results are shown in FIG. 1, FIG. 2 and Table 1.

TABLE 1

| | | | Comparative | Comparative |
|---|---|---|---|---|
| Compound | Example 1 | Example 2 | example 1 | example 2 |
| Absorbance | 309 | 309 | 301 | 312 |
| peak | 373 | 379 | 340 | 355 |
| (nm) | | | | |

As shown in the UV absorption spectra of FIG. 1, compared to the compound of Comparative example 1, the spectra of the compounds of Examples 1 and 2 show significant red shifts at the absorbance peaks. In addition, the positions of the limiting absorbance peaks of the compounds of Examples 1 and 2 have greater wavelengths than those of Comparative examples 1 and 2, and can be extended to 420 nm or more. Moreover, compared to the compound of Comparative example 2 in which no oxygen-containing group is located at the 5-position on the phenyl ring, the compound of Example 2 in which the oxygen-containing group is located at the 5-position on the phenyl ring shows a red shift at the absorbance peaks with greater wavelength (>350 nm), as shown in Table 1. These results indicate that the spectrum of the compound shows the red shift at the absorbance peaks when the sulfur-containing group is located at the 5-position on the benzo ring and the oxygen-containing group is located at the 5-position on the phenyl group.

In addition, as shown in FIG. 2, the compound of Example 2 has the feature that the oxygen atom at the 5-position on the phenyl ring is bound with the alkyl substituted with hydroxyl, and the compound of Example 3 has the feature that the oxygen atom at the 5-position on the phenyl ring is bound with the alkyl substituted with acrylate. The positions of the absorbance peaks of these two compounds are only slightly differed. Furthermore, the compound of Example 2 has the feature that the oxygen atom at the 5-position on the phenyl ring is bound with the alkyl substituted with hydroxyl, and the compound of Example 8 has the feature that the oxygen atom at the 5-position on the phenyl ring is bound with the benzyl group. The positions of the absorbance peaks of these two compounds are almost the same. These results indicate that the different reactive functional groups bound to the oxygen atoms at the 5-position on the phenyl ring do not make great influence on the absorbance peaks on the UV spectra. It is because these reactive functional groups are not directly bound to the conjugated structures of the molecules. The difference is mainly caused by the strength changes of the absorbance, which are resulted from the difference of the molecular weights and the interaction between the solvents.

### Test Example 2: Transmission Test

The compound of Example 2 or the compound of Comparative example 1 (0.05 g) was mixed with the polyurethane (PU) main agent (Item number: A-7121) (4 g) and ethyl acetate (2 g), followed by mixing with the curing agent (Item number: Bayer N75) (2 g). After mixing, the obtained PU glue was applied onto a PET film with a thickness of 125 μm by using a coating machine and a coating rod. After the coating process, the obtained wet film had a thickness of 100 μm. After drying at 80° C. for 30 min, the transmittance of the obtained film was measured. Herein, the transmittance of the PU film was calculated to exclude the absorbance of the PET film.

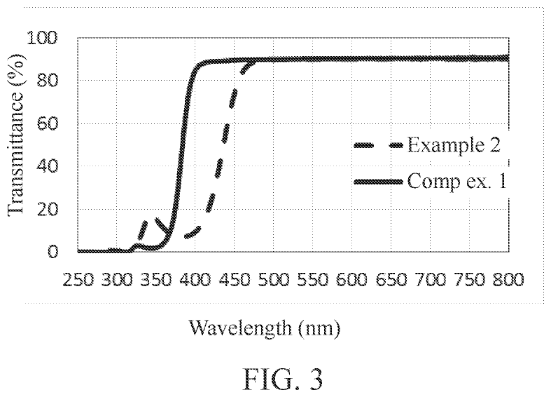

As shown in the results of FIG. 3, the spectrum of the compound of Example 2 shows the red shift, and the compound of Example 2 can absorb light having wavelengths within 400 nm to 450 nm. The results shown in FIG. 3 also indicates that the position of the limiting absorbance peak of the compound of Comparative example 1 is located at around 400 nM, but that of the compound of Example 2 is located at 450 nm or more. The aforesaid results indicate that the spectrum of the compound of Example 2 shows significant red shifts compared to the spectrum of the compound of Comparative example 1, and these results are consistent with the results measured in the solution form.

### Test Example 3: Extraction Test

The compound of Example 2 or the compound of Comparative example 1, 3 or 4 (0.05 g) was mixed with the polyurethane (PU) main agent (Item number: A-7121) (6 g) and the solvent (3 g), followed by mixing with the curing agent (Item number: Bayer N75) (3 g). The mixture was stirred and placed at room temperature for 4 hr, followed by drying in an oven at 80° C. for 16 hr. Then, the solvent (30 g) was added, followed by extracting for 2 hr by using the ultrasonicator. After the extraction, the extraction rate was measured. Herein, the solvent for the compound of Comparative example 3 was a mixing solvent of ethyl acetate/toluene (weight ratio of ethyl acetate to toluene=1:2), and the solvent for the rest of the compounds was ethyl acetate. The results are shown in the following Table 2.

TABLE 2

| Compound | Molecular weight | Extraction rate |
|---|---|---|
| Example 2 | 449.5 | 0%* |
| Comparative example 1 | 451.6 | 99% |
| Comparative example 3 | 658.9 | 73% |
| Comparative example 4 | 479.6 | 94% |

*0%, which means the compound was not found.

The results shown in FIG. 2 indicate that the reactive compound of Example 2 has better extraction resistance than the unreactive compound of Comparative example 1 even though these two compounds have similar molecular weight. The compound of Example 2 cannot be found after the extraction test, which means the compound of Example 2 has significant extraction resistance. However, the compound of Comparative example 1 was almost extracted out. In addition, compared to the compound of Comparative example 3 with larger molecular weight, the compound of Example 2 shows excellent extraction resistance. However, more than 70% of the compound of Comparative example 3 was extracted out. Furthermore, compared to the compound of Comparative example 4, the compound of Example 2 also shows better extraction resistance.

### Test Example 4: Extraction Test

The compound of Example 3 or the compound of Comparative example 1 (0.03 g) was added into a mixture of 1,6-Hexanediol diacrylate (HDDA) (0.9 g), tetrahydrofurfuryl acrylate (THFA) (1.0 g) and 1,1′-Azobis(cyclohexanecarbonitrile) (ABCN) (0.07 g). After stirring and mixing, the mixture was dried in an oven at 80° C. for 24 hr. Then, ethyl acetate (10 g) was added, followed by extracting for 2 hr by using the ultrasonicator. After the extraction, the extraction rate was measured. The results are shown in the following Table 3.

TABLE 3

| Compound | Molecular weight | Extraction rate |
|---|---|---|
| Example 3 | 585.7 | 0.5% |
| Comparative example 1 | 451.6 | 93.2% |

The results shown in Table 3 indicate that the reactive compound of Example 3 has better extraction resistance than the unreactive compound of Comparative example 1. Only a small amount of the compound of Example 3 was extracted out after the extraction test, which means the compound of Example 3 has significant extraction resistance. However, the compound of Comparative example 1 was almost extracted out.

In conclusion, the spectra of the novel compounds provided by the present disclosure show red shifts at the absorbance peaks. When the novel compounds provided by the present disclosure are applied onto a substrate, which is sensitive to electromagnetic radiation with a wavelength greater than 380 nm, to form a coating layer, the obtained coating layer can effectively absorb the light having wavelength greater than 380 nm. Thus, the novel compound provided by the present disclosure can be used for forming an anti-blue light or anti-UV coating layer, to provide a product with anti-blue light or anti-UV effect, such as a protection film, glasses lens, contacts or intraocular lens. In addition, the novel compound provided by the present disclosure further has extraction resistance. When the novel compound provided by the present disclosure is polymerized with monomer or oligomer to form a polymer with UV absorbers, the application of the obtained polymer can be extended because the UV absorbers contained in the obtained polymer have excellent extraction resistance.

Although the present disclosure has been explained in relation to its embodiment, it is to be understood that many other possible modifications and variations can be made without departing from the spirit and scope of the disclosure as hereinafter claimed.

What is claimed is:

1. A compound represented by the following formula (I): 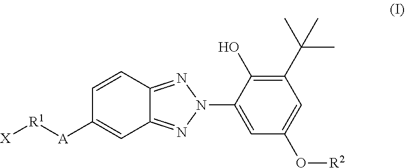
wherein,
A is —S— or —SO₂—;
R¹ is straight or branched C₁₋₁₀ alkylene, straight or branched C₁₋₁₀ alkylene substituted by —OH, or straight or branched C₁₋₁₀ alkylene interrupted by an ester group;
R² is —R³Y, H, straight or branched C₁₋₁₀ alkyl, or C₆₋₁₅ aralkyl;
R³ is straight or branched C₁₋₁₀ alkylene, straight or branched C₁₋₁₀ alkylene substituted by —OH, or straight or branched C₁₋₁₀ alkylene interrupted by an ester group;
each of X and Y respectively is —OH, —OCOR⁴, —NH₂, —NCOR⁴, —NCO, 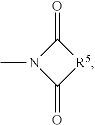
—CO₂H, —CO₂R⁴, 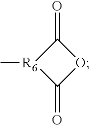
R⁴ is straight branched C₁₋₁₀ alkyl, or straight or branched C₃₋₁₀ alkenyl;
R⁵ is C₃₋₁₀ cycloalkenylene; and
R⁶ is straight or branched C₁₋₁₀alkylene, or 1,2-phenylene.

2. The compound of claim 1, wherein each of X and Y respectively is —OH, —OCOR⁴, —NH₂, 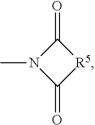
—CO₂R⁴, or 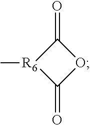
R⁴ is straight or branched C₁₋₆ alkyl, or straight or branched C₃₋₆ alkenyl; R⁵ is C₃₋₁₀ cycloalkenylene; and R⁶ is straight or branched C₁₋₆ alkylene.

3. The compound of claim 2, wherein each of X and Y respectively is —OH, —OCOR^{4a}, —NH₂, 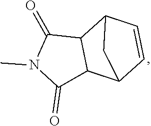
—CO₂R^{4b} or 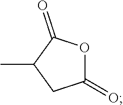
R^{4a} is straight or branched C₃₋₆ alkenyl; and R^{4b} is straight or branched C₁₋₆ alkyl.

4. The compound of claim 1, wherein A is —S—; R¹ is straight or branched C₁₋₁₀ alkylene, or straight or branched C₁₋₁₀ alkylene substituted by —OH; X is —OH, 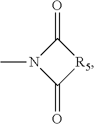
or —CO₂R⁴; R⁴ is straight or branched C₁₋₁₀ alkyl; and R⁵ is C₃₋₁₀ cycloalkenylene.

5. The compound of claim 4, wherein R¹ is straight or branched C₁₋₆ alkylene, or straight or branched C₁₋₆ alkylene substituted by —OH; X is —OH, 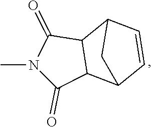
or —CO₂R⁴; R⁴ is straight or branched C₁₋₆ alkyl.

6. The compound of claim 1, wherein A is —SO₂—; R¹ is straight or branched C₁₋₁₀ alkylene, or straight or branched C₁₋₁₀ alkylene interrupted by an ester group; X is —OH, —OCOR⁴, —NH₂ or 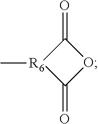
R⁴ is straight or branched C₃₋₁₀ alkenyl; and R⁶ is straight or branched C₁₋₁₀ alkylene.

7. The compound of claim 6, wherein R¹ is straight or branched C₁₋₆ alkylene, or straight or branched C₁₋₆ alkylene interrupted by an ester group; X is —OH, —OCOR⁴, —NH₂ or 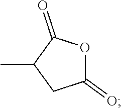
and R⁴ is straight or branched C₃₋₆ alkenyl.

8. The compound of claim 1, wherein R² is H, straight or branched C₁₋₆ alkyl, or benzyl.

9. The compound of claim 1, wherein R² is —R³Y; R³ is straight or branched C₁₋₁₀ alkylene, or straight or branched C₁₋₁₀ alkylene interrupted by an ester group; Y is —OH, —OCOR⁴, —NH₂, 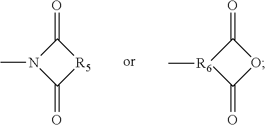
R⁴ is straight or branched C₃₋₁₀ alkenyl; R⁵ is C₃₋₁₀ cycloalkenylene; and R⁶ is straight or branched C₁₋₁₀ alkylene.

10. The compound of claim 9, wherein R³ is straight or branched C₁₋₆alkylene, or straight or branched C₁₋₆alkylene interrupted by an ester group; Y is —OH, —OCOR⁴, —NH₂, 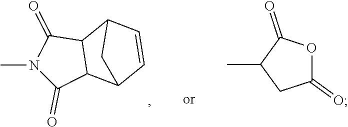
and R⁴ is straight or branched C₃₋₆ alkenyl.

11. The compound of claim 1, which is any one of the following formulas (I-1) to (I-13):

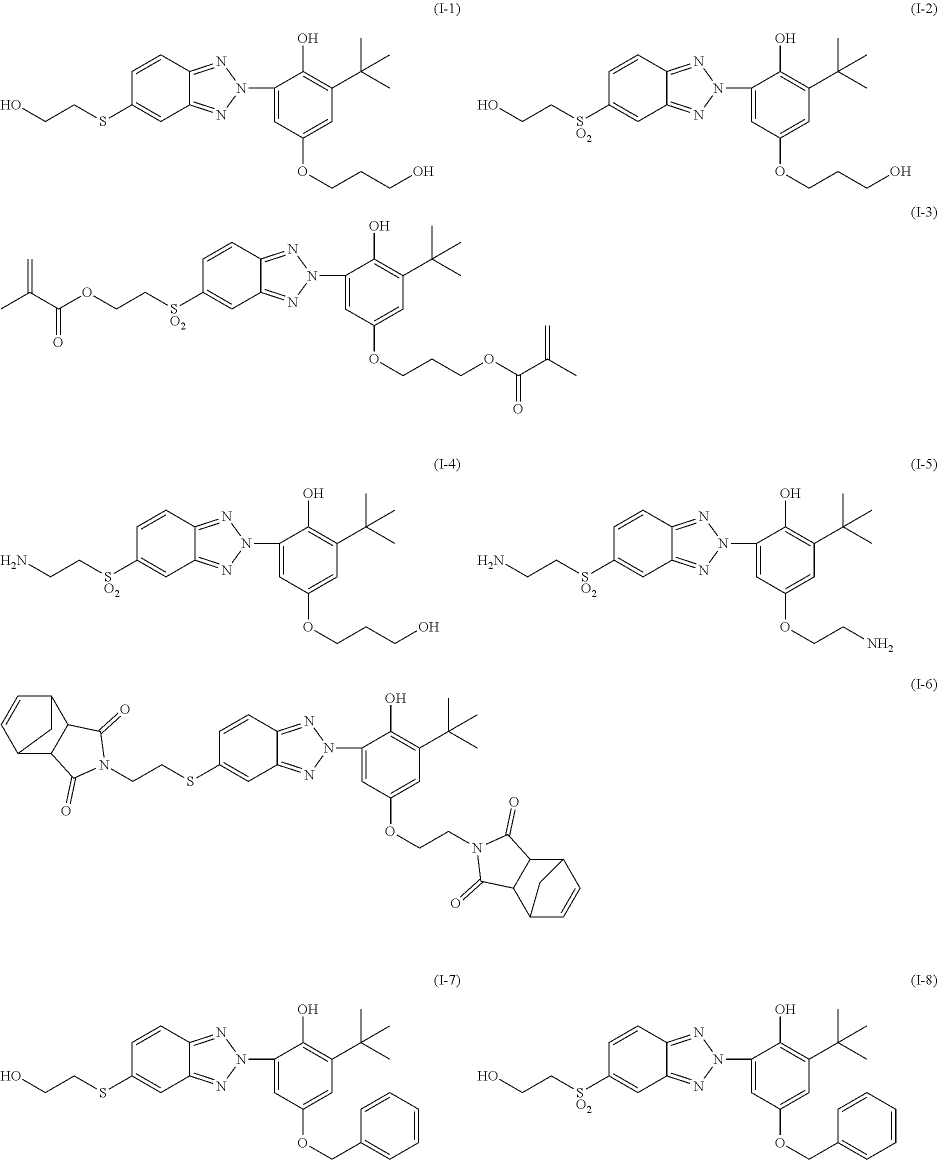

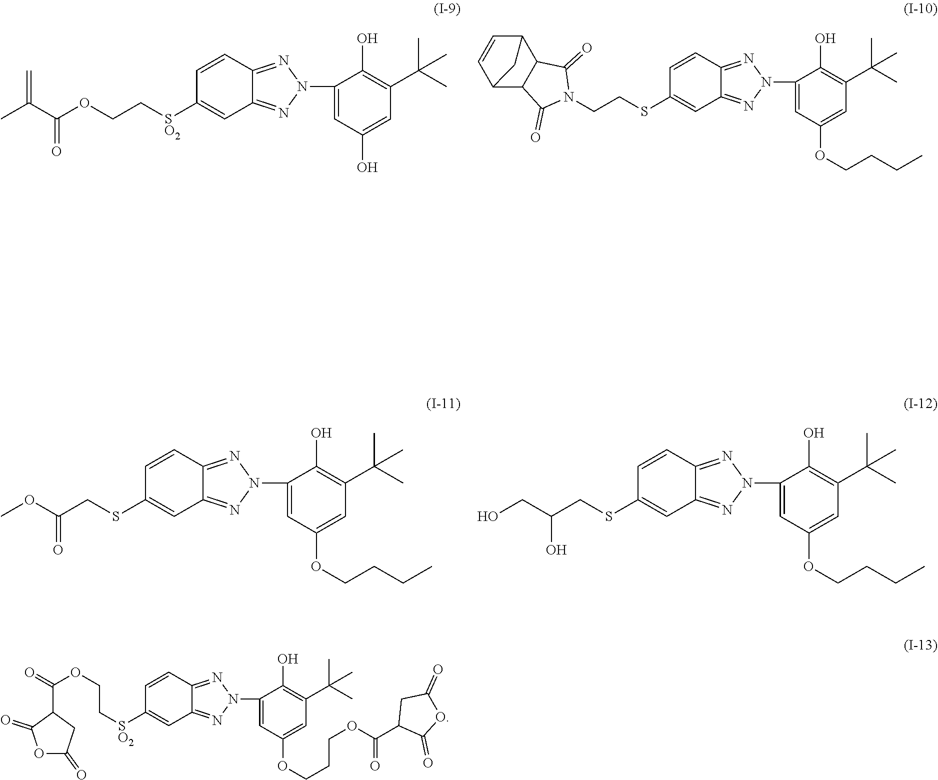

12. A composition with stability to photo-induced degradation including:
(A) a photo-induced degradable organic material; and
(B) a compound represented by the following formula (I): 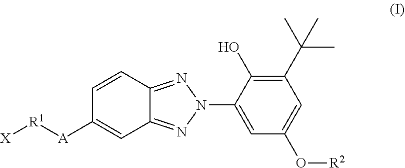
wherein,
A is —S— or —SO₂—;
R¹ is straight or branched C₁₋₁₀ alkylene, straight or branched C₁₋₁₀ alkylene substituted by —OH, or straight or branched C₁₋₁₀ alkylene interrupted by an ester group;
R² is —R³Y, H, straight or branched C₁₋₁₀ alkyl, or C₆₋₁₅ aralkyl;
R³ is straight or branched C₁₋₁₀ alkylene, straight or branched C₁₋₁₀ alkylene substituted by —OH, or straight or branched C₁₋₁₀ alkylene interrupted by an ester group;
each of X and Y respectively is —OH, —OCOR⁴, —NH₂, —NCOR⁴, —NCO, 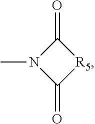
—CO₂H, —CO₂R⁴, 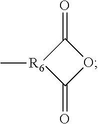
R⁴ is straight or branched C₁₋₁₀ alkyl, or straight or branched C₃₋₁₀ alkenyl;
R⁵ is C₃₋₁₀ cycloalkenylene; and
R⁶ is straight or branched C₁₋₁₀ alkylene, or 1,2-phenylene.

13. The composition of claim 12, which is used for forming a coating layer.

14. The composition of claim 13, wherein the coating layer is formed on a substrate which is sensitive to electromagnetic radiation with a wavelength greater than 380 nm.

15. The composition of claim 12, wherein a content of the compound is 0.1% to 30% based on a weight of the photo-induced degradable organic material.

16. A glasses lens with anti-blue light effect, comprising a compound represented by the following formula (I): 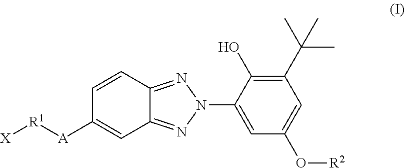
wherein,
A is —S— or —SO₂—;
R¹ is straight or branched C₁₋₁₀ alkylene, straight or branched C₁₋₁₀ alkylene substituted by —OH, or straight or branched C₁₋₁₀ alkylene interrupted by an ester group;
R² is —R³Y, H, straight or branched C₁₋₁₀ alkyl, or C₆₋₁₅ aralkyl;
R³ is straight or branched C₁₋₁₀ alkylene, straight or branched C₁₋₁₀ alkylene substituted by —OH, or straight or branched C₁₋₁₀ alkylene interrupted by an ester group;
each of X and Y respectively is —OH, —OCOR⁴, —NH₂, —NCOR⁴, —NCO, 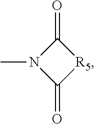
—CO₂H, —CO₂R⁴, 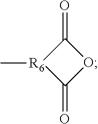
R¹ is straight or branched C₁₋₁₀ alkyl, or straight or branched C₃₋₁₀ alkenyl;
R⁵ is C₃₋₁₀ cycloalkenylene; and
R⁶ is straight or branched C₁₋₁₀ alkylene, or 1,2-phenylene.

17. A protection film with anti-blue light effect, comprising a compound represented by the following formula (I): 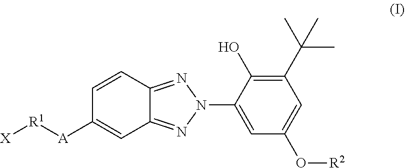
wherein,
A is —S— or —SO₂—;
R is straight or branched C₁₋₁₀ alkylene, straight or branched C₁₋₁₀ alkylene substituted by —OH, or straight or branched C₁₋₁₀ alkylene interrupted by an ester group;
R² is —R³Y, H, straight or branched C₁₋₁₀ alkyl, or C₆₋₁₅ aralkyl;
R³ is straight or branched C₁₋₁₀ alkylene, straight or branched C₁₋₁₀ alkylene substituted by —OH, or straight or branched C₁₋₁₀ alkylene interrupted by an ester group;
each of X and Y respectively is —OH, —OCOR⁴, —NH₂, —NCOR⁴, —NCO, 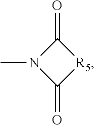
—CO₂H, —CO₂R⁴, or 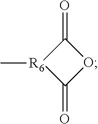
R⁴ is straight or branched C₁₋₁₀ alkyl, or straight or branched C₃₋₁₀ alkenyl;
R⁵ is C₃₋₁₀ cycloalkenylene; and
R⁶ is straight or branched C₁₋₁₀ alkylene, or 1,2-phenylene.
